(19) United States
(12) Patent Application Publication
(10) Pub. No.: US 2005/0118731 A1
(43) Pub. Date: Jun. 2, 2005

(54) Method and apparatus using a surface-selective nonlinear optical technique for detection of probe-target interactions without labels

(76) Inventor: Joshua S. Salafsky, New York, NY (US)

Correspondence Address: Gordon & Jacobson, P.C., 65 Woods End Road, Stamford, CT, 06905, US

(21) Appl. No.: 10/970,754

(22) Filed: Oct. 21, 2004

Related U.S. Application Data
(63) Continuation-in-part of application No. 09/907,038, filed on Jul. 17, 2001, now abandoned.
(60) Provisional application No. 60/260,261, filed on Jan. 8, 2001.
(60) Provisional application No. 60/260,300, filed on Jan. 8, 2001.
(60) Provisional application No. 60/262,214, filed on Jan. 17, 2001.

(51) Int. Cl. G01N 33/543

(52) U.S. Cl. 436/518

(57) ABSTRACT

A surface-selective nonlinear optical technique, such as second harmonic or sum frequency generation, is used to detect target-probe binding reactions or their effects, at an interface, in the presence of indicators. In addition, the direction of the nonlinear light is scattered from the interface in a well-defined direction and therefore its incidence at a detector some distance from the interface may be easily mapped to a specific and known location at the interface.

### RELATED APPLICATIONS

This application is a continuation-in-part of U.S. Ser. No. 09/907,038, filed Jul. 17, 2001, which claims the benefit of U.S. provisional applications No. 60/260,261, entitled, “Method and Apparatus Using a Surface-Selective Nonlinear Optical Technique for Detection of Probe-Target Interactions Without Labels”, filed, Jan. 8, 2001; No. 60/260,300, entitled “Apparatus and Method for the Detection of Biological Reactions Using a Surface-Selective Nonlinear Optical Technique and an Indicator, filed Jan. 8, 2001; and No. 60/262,214, entitled “Method and Apparatus Using a Surface-Selective Nonlinear Optical Technique for Detection of Probe-Target Interactions Without Labels”, filed Jan. 17, 2001, all of which are incorporated by reference herein in their entireties.

### FIELD OF THE INVENTION

The present invention discloses methods and various configurations of an apparatus for detecting reactions between biological components, or the effects of the reactions, at an interface and without the need for labeling the components. The present invention also discloses methods and an apparatus for imaging biological components at various surfaces. Indicators are used for detecting or imaging the nonlinear optical response at an interface.

### BACKGROUND OF THE INVENTION

Detecting and quantifying binding between biomolecules is of central interest in modern molecular biology and medicine. Genomics and proteomics research is increasingly directed toward this problem, which demands high-throughput analysis of a variety of biological interactions. Many schemes for doing this rely on immobilization of molecules, often oligonucleotides or proteins, to solid surfaces. In particular, a microarray format of samples can be used to obtain information in a highly parallel process. For example, Fodor et al. (1991—relevant portions of which are incorporated by reference herein) disclose high density arrays formed by light-directed synthesis—in this case, the surface-attached probes are oligonucleotides and are tested for binding (hybridization) against targets. The targets, freely diffusing in solution, are fluorescently-labeled oligonucleotides and at places where the nucleotide sequence of the probe matches the sequence of the target, binding occurs. When non-bound targets are removed by washing, the sequence of the remaining targets can be determined by scanning the surface for fluorescence since the probe sequence is known, by design, at each location on the surface, and targets and probes must have matching, complementary sequences to hybridize. A number of variations on this method have been introduced including studying SNPs (single nucleotide polymorphisms), where the binding strength, and hence the fluorescence intensity, between sequences differing by one base-pair; detection of protein-protein interactions, where one protein (the probe) is immobilized to the surface and tested for binding against a variety of targets; protein-drug interactions where protein-protein interactions are modulated by the presence of a drug; and others.

In all these cases, the read-out step involves fluorescence-based detection. However, detection with fluorescence has several drawbacks: the use of labels introduces additional time and cost, the samples are generally dry (to remove background fluorescence; i.e., non-bound targets in the bulk) and therefore no equilibrium (free energy, dissociation constant, etc.) measurement can be easily made due to fluorescence background from the bulk. The non-bound targets must first be removed from the sample via a wash step and this obviates equilibrium or kinetics measurements and, furthermore, can be time-consuming when many scans must be made on a given sample or many samples must be examined. The excitation source for fluorescence may also contribute a background signal since it can be scattered by the substrate into the detection optics, and may be difficult to completely filter from the fluorescence. Furthermore, there may be background autofluorescence or bottlenecks in the “read-out” or detection step because the scan can require pixel-by-pixel acquisition, as for example with confocal-based detection schemes. Auto-focusing routines at each step in the scan can also lead to significant slow-down in image acquisition.

Another method for quantifying biomolecular binding interactions—with an ability to measure both equilibrium and kinetics properties of the interaction is surface plasmon resonance (SPR). SPR requires a conductive or semiconductive layer (typically gold) between the substrate (typically glass) and the liquid solution it is immersed in. Incident light is coupled into the conductive layer by means of a prism or grating and, at a specific wavelength or angle of incidence, a resonance occurs, resulting in a sharp minimum or decrease in reflectivity. Generally, a bio-compatible layer or layers are built on top of the conductive layer. In one example, proteins are immobilized to the biocompatible layer (often dextran-based) and target proteins are brought into contact with the layer. The resonance wavelength or angle depends on the refractive index of the solution near the substrate and this, in turn, depends on the amount and mass of adsorbed biomolecules within an evanescent wavelength from the conductive layer. When target protein binds to the immobilized protein, a change in the resonance wavelength or angle occurs. However, the SPR technique is not convenient for detecting samples in an array format because of the difficulty in coupling the excitation into each array element separately. Furthermore, the detection sensitivity may be low, the technique cannot distinguish between specific and non-specific binding, and SPR typically requires an extra, biologically compatible layer to prevent destructive interactions which can occur if the biomolecules make contact with the conductive layer. This biocompatible layer may not always be stable or prevent destructive interactions with the gold surface and the immobilized proteins must often be truncated in order to render them suitable for coupling to the bio-compatible layer, thus risking the possibility that their properties may change. A particularly acute problem occurs with membrane proteins. Membrane proteins are best studied in a native-like environment such as a laterally fluid phospholipid membrane which can be prepared on glass surfaces. However, it is not possible to prepare these membranes on gold surfaces due to destructive interactions between the gold and the lipids.

Surface-selective nonlinear optical (SSNLO) techniques such as second harmonic generation (SHG) allow one to detect interfacial molecules or particles (the interface must be non-centrosymmetric) in the presence of the bulk species. An intense laser beam (the fundamental) is directed on to the interface of some sample; if the interface is non-centrosymmetric, the sample is capable of generating nonlinear light, i.e. the harmonics of the fundamental. The fundamental or the second harmonic beams can easily be separated from each other, unlike the typical case in fluorescence techniques with excitation and emission light, which are separated more narrowly by the Stokes shift. Individual molecules or particles can be detected if they 1) are nonlinearly active (possess a hyperpolarizability) and 2) are near to the surface and through its influence (via chemical or electric forces) become non-randomly oriented. This net orientation and the intrinsic SHG-activity of the species are responsible for an SHG-allowed effect at the interface. For example, the adsorption processes of dye molecules to planar solid surfaces (glass and silica), liposomes and solid beads (silica and polystyrene) at air-water interface have been measured. The technique has also been used to follow such processes as electron-transfer or solvation dynamics at an interface.

Nonlinear SSNLO techniques, such as SHG, have previously been confined mainly to physics and chemistry since relatively few biological samples are intrinsically non-linearly active. Examples include the use of an optically nonlinear active dye that is used to image biological cells (Campagnola et al., Peleg et al.). In this art, nonlinear active stains are immobilized in membranes and these stains are used to image the cell surfaces. However, the stains intercalate into the membranes in either an ‘up’ or ‘down’ direction, thus reducing the total nonlinear signal due to destructive interference. Nonlinear optically active dyes have also been used to measure the kinetics of those dyes crossing lipid bilayers in liposomes (Srivastava and Eisenthal). Recently, too, in the art, the concept and technique of second harmonic active labels (“SHG labels”) was introduced, allowing any non-linear active molecule or particle to be rendered non-linear active (Salafsky). The first example of this was demonstrated by labeling the protein cytochrome c with an oxazole dye and detecting the protein conjugate at an air-water interface with second harmonic generation.

### SUMMARY OF THE INVENTION

The present invention is based on both the surface-selectivity of second harmonic (or sum/difference frequency) generation and the nonlinear-active properties of indicators polarized or oriented near a charged surface. In addition, because the nonlinear beam (e.g., second harmonic) is scattered from an interface in a well-defined direction—in contrast to fluorescence detection in which fluorescence is emitted nearly isotropically—this lends itself to imaging techniques or the use of arrays. Surface-selective nonlinear optical techniques are coherent techniques—the fundamental and nonlinear beams have well-defined phase relationships, and the propagating wavefronts of a nonlinear beam in a macroscopic sample are in phase (within the coherence length). These properties offer a number of advantages useful for surface or high-throughput studies in which, for example, a microarray surface is studied. An apparatus using nonlinear optical suface-selective-based detection, such as second harmonic generation, requires minimal collection optics since generation of the nonlinear light only occurs at the interface and thus affords extremely high depth discrimination and fast scanning.

Furthermore, the binding process between probes and targets is detected without the need for labels, via the indirect effect the binding process has on the surface electric charge and potential and, in turn, the polarization of indicators near the interface, and this results in a time and cost-savings compared to methods which require labels. SSNLO can also lead to much higher signal to noise of detection than can techniques using fluorescence-based detection.

Indicators include nonlinear-active molecules that possess a hyperpolarizability, or particles whose nonlinear optical properties or orientation near a surface or interface is modulated as the electric charge polarization, charge density or potential of the surface is modulated. In one aspect of the invention, the charge or potential of an interface is modulated by the binding of a target to probes immobilized on the surface. In another aspect, the surface electric potential of a cell is changed by a change in the ion channel properties—an opening, closing, increase or decrease in ionic permeability in response to target (ligand) binding, for instance. In another aspect of the invention, an indicator serves as a marker for imaging purposes, e.g., to image cells or tissues. An indicator preferably does not appreciably alter or participate in the target-probe reaction itself. The indicator can be dissolved or suspended in the liquid, medium, solution or aqueous phase containing the target component. An indicator preferably does not translocate into the lipid bilayer of vesicles or cells. An indicator preferably possesses freedom of movement to respond to changes in surface electric charge density or potential.

### BRIEF DESCRIPTION OF THE DRAWINGS

FIG. 1 depicts one embodiment of the apparatus in which the mode of generation and collection of the second harmonic light is by reflection off the substrate with surface-attached probes.

FIG. 2 depicts one embodiment of an apparatus in which the mode of generation and collection of the second harmonic light is by total internal reflection through a prism. The prism is coupled by an index-matching material to a substrate with surface-attached probes.

FIG. 3 depicts one embodiment of an apparatus in which the mode of generation and collection of the second harmonic light is by total internal reflection through a wave-guide with multiple reflections as denoted by the dashed line inside the wave-guide.

FIGS. 4A-C depict one embodiment of a flow-cell for delivery and removal of biological components and other fluids to the substrate containing attached probes.

FIG. 5A-C depict three embodiments of an apparatus in which the mode of generation and collection of the second harmonic light is by transmission through a sample. In FIG. 5A, the second harmonic beam is co-linear with the fundamental. In FIG. 5B, the second harmonic is collected from a direction orthogonal to the fundamental (‘right-angle collection’). In FIG. 5C, the second harmonic light is collected by an integrating sphere and a fiber optic line.

FIG. 6 depicts an embodiment of the transformation, using a series of optical components, of a collimated beam of the fundamental light into a line shape suitable for scanning a substrate.

FIG. 7A depicts an embodiment of a substrate surface (containing attached probes) which has been patterned into an array format (elements 1-35).

FIG. 7B depicts one element of a substrate array in which each element is a well with walls, with surface-attached probes, and the well is capable of holding some liquid and serves to physically separate the well's contents from adjacent wells or other parts of the substrate.

FIG. 8 depicts one embodiment of a surface chemistry used to attach oligonucleotide or polynucleotide samples to the substrate surface.

FIG. 9A depicts an embodiment of a substrate containing multiple wells (1-16), each of which contains surface-attached probes as depicted in FIG. 9B.

FIG. 10 depicts an embodiment of the apparatus substrate with the use of an aminosilane surface-attached layer on top of a reflective coating. The reflective coating underneath the aminosilane layer improves collection of the nonlinear optical light. The aminosilane layer is suitable for coupling biomolecules or other probe components to the substrate.

FIGS. 11A and B depict an embodiment of an apparatus in which the mode of generation and collection of the second harmonic light is through a fiber optic. FIG. 11A depicts the use of a bundle of fiber optic lines, and FIG. 11B depicts the use of beads coupled to the end of a fiber for attaching probes.

FIGS. 12A-C depict three embodiments of an apparatus in which the mode of generation and collection of the second harmonic light is by transmission through a sample. FIG. 12A depicts both the fundamental and second harmonic beams travelling co-linearly through a sample. FIG. 12B depicts the fundamental and second harmonic beams being refracted at the top surface (top surface contains attached probes) of a substrate with this surface generating the second harmonic light. FIG. 12C depicts a similar apparatus to FIG. 12B except that the bottom surface (bottom surface contains attached probes) generates the second harmonic light.

FIGS. 13A-B depict two embodiments of an apparatus in which second harmonic light is generated by total internal reflection at an interface. The points of generation of the second harmonic light are denoted by the circles. In FIG. 13A, a dove prism is used to guide the light to a surface capable of generating the second harmonic light (bottom surface of prism but can also be another surface coupled to the prism through an index-matching material). In FIG. 13B, a wave-guide structure is used to produce multiple points of second harmonic generation.

FIGS. 14A-C depicts three embodiments of an apparatus in which second harmonic light is generated using a fiber optic line (with attached probes at the end of the fiber). FIG. 14A depicts an apparatus in which both generation and collection of the second harmonic light occur in the same fiber. FIG. 14B depicts the use of a bead containing surface-attached probes at the end of the fiber. FIG. 14C depicts an apparatus in which the second harmonic light is generated at the end of the fiber optic (containing attached probges) and collected using a mirror or lens external to the fiber optic.

FIGS. 15A-B depict two embodiments of an apparatus using an optical cavity for power build-up of the fundamental.

FIG. 16A-C depict three embodiments of an apparatus in which the mode of generation and collection of the second harmonic light uses reflection of the light from an interface.

### DETAILED DESCRIPTION OF THE INVENTION

The following describes methods and concepts using second harmonic generation, but these apply equally to any nonlinear surface-selective technique. Second harmonic (SH) spectroscopy is a non-linear, surface-selective technique for detecting molecules within a molecularly thin layer near a surface (Eisenthal, 1996; Corn and Higgins, 1994). The molecules are oriented by the surface through chemical or electrostatic forces and irradiation with a fundamental beam (2.omega.) leads to the generation of second harmonic (.beta.) light; molecules in the bulk, which are randomly oriented, produce no SH light. Generally, SH studies of molecules involve species with a large molecular hyperpolarizability (p) detected in a resonantly enhanced process. However, it has been shown that water at a charged interface, silica for example, produces an SH signal, due to both the monomolecular water layer oriented directly at the interface and a longer-range contribution due to the water molecules becoming polarized by the static electric field of the charged surface (Ong et al, 1992; Zhao et al, 1993).

The theoretical background of the present invention can be described, but is not limited by, the following. The production of SH light by water at a charged interface can be described by the following equation:
√ISH.varies.E.sub.2.omega.=A.chi.sup.(2)+B.PHI..sub.0.chi..s- up.(3) (1)
where I.sub.SH is the SH intensity, E is the electric field of the SH light, .chi..sup.(2) and .chi..sup.(3) are the second and third-order nonlinear susceptibilities, PHI. is the surface potential, and A and B are constants which depend on the specific properties of the surface (Ong et al, 1992). In effect, the total SH light generated is due to both a monomolecular surface contribution (.chi..sup.(2) part) and that due to the polarization of water molecules by the electric field of the charged surface (.chi..sup.(3) part). The electric potential of a charged surface in contact with electrolyte, and its decay to the bulk value follows the Gouy-Chapman model
   where: 1=11.7C sin h(19.5z0)tanh (ze/4kT)tan h(ze0/4 kT)=-x (2)
with sigma. the surface charge density (.mu.C/cm.sup.2), C the bulk electrolyte concentration, z the charge of the electrolyte, PHI..sub.0 the surface potential, .PHI. the potential at a distance x from the surface, and .kappa. the Debye length at 25.degree. (Bard and Faulkner, 1980). As shown in the art, when charged protein is adsorbed to a charged surface, the surface potential (.PHI..sub.0) is modulated by the charged protein; this, in turn affects the amount of water oriented by the surface and therefore the second harmonic signal. The effect can be monitored with high sensitivity and in real time to measure Langmuir adsorption isotherms, kinetics, and surface densities of the protein.

In addition to water being polarized near a charged interface, solvent or indicator molecules or particles can be polarized. Indicators possess a hyperpolarizability and their polarization or orientation near a charged interface can be modulated as the surface charge changes; they can be dissolved or suspended in the medium; they do not participate appreciably in probe-target reactions. The use of indicators can boost the nonlinear response of a measurement if their hyperpolarizability and polarization response is higher than that of water or other solvent molecules. Given a surface potential and its corresponding electric field (E*), and the following expression for the mean dipole moment:
.mu..sub.mean=.mu.sup..2E*/3kT (3)
with .mu. the ground-state dipole moment of water (1.85/D), solvent or indicators, and kT the Boltzmann constant multiplied by the temperature (K), the number of water molecules, solvent or indicators oriented by the surface can be calculated. This number is L=.mu..sub.mean/.mu. where L is the Langevin function. For example, given a typical surface potential of 100 mV and an electric field of order 10.sup.6 V/cm near the interface, one can calculate that .about. 10.sup. 16 water molecules are oriented (about 10%) within the volume experiencing a surface polarization. The intensity of second harmonic light generated by the oriented water molecules is quadratic in their number and proportional to the hyperpolarizability. It is of advantage to produce the highest nonlinear optical signal possible, both for considerations of the signal-to-noise of the technique and its sensitivity.

The present invention is concerned with interactions between molecules, such as a binding reaction or a conformational change. A binding reaction between probes' surfaces (or surface-attached probes) and targets modulates the surface electric charge density or potential. SSNLO techniques can be used to measure these interactions via the effect the surface charge density or potential has on indicators near the interface. Because most biological components, such as proteins, viruses and nucleic acids, possess a net charge, the surface potential will be altered when binding to a complementary probe component at an interface (via Eqn. 2). The alteration in surface potential will, in turn, alter the polarization or orientation of the indicators near the surface and the second harmonic signal that the interface produces (via Eqn. 1). In general, the indicator molecule, moiety or particle can be dissolved or suspended in the solution or aqueous phase containing the surface to be imaged or the phase containing target components (and should not appreciably alter or participate in the target-probe reaction). Furthermore, interactions can comprise a conformational change, such as a ligand-induced conformational change with a GPCR such as the beta-2 receptor.

The invention discloses a method for screening one or more candidate binding partners (referred to herein as probes) for binding to a test molecule (referred to herein as targets). The method involves illuminating a sample with one or more light beams at one or more fundamental frequencies, said sample comprising the test molecule exposed to the one or more candidate binding partners, and measuring one or more physical properties of a nonlinear optical light beam emanating from the sample. A change in the value of the one or more physical properties measured relative to a value for the one or more physical properties measured in the absence of exposure of said test molecule to said one or more candidate binding partners indicates that said one or more candidate binding partners bind said test molecule.

The invention discloses a method for screening one or more candidate modulator molecules for the ability to modulate an interaction between a test molecule and its binding partner. The method involves illuminating a sample with one or more light beams at one or more fundamental frequencies, said sample comprising said test molecule exposed to (i) said binding partner, and (ii) said one or more candidate modulator molecules, and measuring one or more physical properties of a nonlinear optical light beam emanating from said sample. A change in the value of said one or more physical properties measured relative to the value for said one or more physical properties measured in the absence of exposure to said one or more candidate modulator molecules indicates that said one or more candidate modulator molecules modulate the interaction between said test molecule and its binding partner.

The invention provides a method for detecting a conformational change in a test molecule upon binding of the test molecule to a binding partner comprising contacting said test molecule with one or more candidate binding partners, where the test molecule or the one or more candidate binding partners is labeled with a nonlinear-active moiety that is not native to the test molecule or the one or more candidate binding partners, respectively. The method involves illuminating said contacted test molecule with one or more light beams at one or more fundamental frequencies, and measuring one or more physical properties of a nonlinear optical light beam emanating from said sample. A change in the value of said one or more physical properties measured relative to the value for said one or more physical properties measured in the absence of said one or more candidate binding partners indicates that at least one of said one or more candidate binding partners bind to said test molecule and that said binding induces a conformational change in said candidate binding partners, in said test molecule, or in both said candidate binding partners and said test molecule.

The invention provides a method for detecting the degree or extent of the binding interaction between a test molecule and one or more binding partner comprising contacting said test molecule with one or more candidate binding partners, wherein the test molecule or the one or more candidate binding partners is labeled with a nonlinear-active moiety that is not native to the test molecule or the one or more candidate binding partners, respectively. The method involves illuminating said contacted test molecule with one or more light beams at one or more fundamental frequencies, and measuring one or more physical properties of a nonlinear optical light beam emanating from the sample. A change in the value of said one or more physical properties measured relative to the value for the one or more physical properties measured in the absence of said one or more candidate binding partners indicates that at least one of said one or more candidate binding partners binds to said test molecule, the degree or extent of the conformational change that said binding induces.

In a specific embodiment, the present invention relates to a method for detecting interactions between biological components using a surface-selective nonlinear optical technique. In one aspect of the present invention this relates to detection of binding between probes and targets that results in a conformational change. The invention discloses methods for screening one or more probes through the conformational changes they induce on binding targets. The invention also discloses methods for screening modulators of the probe-target binding interaction, and for determining the degree or extent of binding through the conformational changes the binding induces.

In summary, probe-target reactions (such as a binding reaction), or the effects of these reactions, causes a change in the electric charge density or potential at an interface. The nonlinear optical response of indicators can be measured to correlate the change in the properties of the nonlinear light (e.g, change in intensity or wavelength in the second harmonic beam) with the amount or rate of the probe-target binding reactions, or the effects of these reactions. Reactions can be performed in the presence of one or more components (e.g., molecules, drugs, particles, etc.) which modulates (e.g., increases or decreases binding strength or rate of reaction) probe-target reactions or secondary, tertiary, etc. reactions caused by the probe-target reactions. The correlation between properties of the nonlinear optical light and the amount or rate of the probe-target reactions, or their effects, can also be made using any mathematical relation or relations known to relate the surface potential to the measured nonlinear optical properties. Examples of these relations include the Langmuir or modified Langmuir equation, the Gouy-Chapman equation, the Stern-Volmer equation, the modified Gouy-Chapman equation, the Stern equation, etc.

Probes are biological components (cells, nucleic acids, nucleic acid analogs, proteins, etc.) immobilized in some fashion to a solid surface or substrate or embedded in a surface (such as a cell surface or liposome) or comprise a surface as with cells or liposome or are cells, liposomes, beads, particles, etc. freely suspended in a sample cell. In all cases, an interface between one surface and another is a necessary condition for generation of the nonlinear surface-selective light. Targets are freely floating biological components which may bind to the probes in a chemical reaction. The electric charge density, and therefore the potential, of a surface determines the amount of water polarized near the interface; for example, the number of polarized indicators determines the amount of nonlinear light (e.g., second harmonic intensity) generated by the interface. When electrically charged targets, probes or both target and probe are electrically charged or dipolar the probe-target binding reaction can modulate the water or indicator polarization or orientation near the interface and therefore the nonlinear radiation intensity, frequency, etc. Furthermore, any probe-target binding reaction which causes a change in surface electric charge or potential (a cell surface, for instance) can be detected by a SSNLO technique via the indirect effects the surface potential has on indicators. Examples of this include: a charged target binding to an uncharged (or partially charged) probe on a surface; a charged target binding to a charged probe; a dipolar target binding to a charged probe; a dipolar target binding to an uncharged probe, etc. Surfaces which carry an electric charge can be imaged using indicators whose nonlinear optical properties, polarization or orientation is sensitive to the surface charge.

The probes or targets of the present invention that can be used include but are not limited to naturally occurring, artificially altered, or genetically engineered, biological species or non-biological species. The candidates for probes or targets also include but are not limited to one or more of the following components: a nucleic acid, protein, small molecule, organic molecule, biological cell, virus, molecular beacon, liposome, receptor, antibody, agonist, antagonist, inhibitor, hapten, ligand, antigen, oocyte, hormone, protein, peptide, receptor, drug, lipid, ganglioside, enzyme, nucleotide, carbohydrate, cDNA, oligonucleotide, nucleoside, polynucleoside, polynucleotide, lipid, ganglioside, oligosaccharide, peptide nucleic acid (PNA), toxin, nucleic acid analog, ion channel receptor, G coupled-protein receptor. In a specific embodiment, the probes can be patterned in an array format on a substrate or solid surface, with the properties or chemical identity of the probes remaining constant or varying among regions of the array.

The present invention offers a number of advantages useful for surface or high-throughput studies in which either a single surface or a microarray surface is studied. An apparatus using nonlinear optical suface-selective-based detection, such as with second harmonic generation, requires minimal collection optics since generation of the nonlinear light only occurs at the interface and thus, in principle, allows extremely high depth discrimination and fast scanning.

Probe-target interactions (e.g., a binding reaction, a conformational change, etc.) can be correlated with the present invention to the following measurable information, for example:
i) the intensity of the nonlinear or fundamental light.
ii) the wavelength or spectrum of the nonlinear or fundamental light.
iii) position of incidence of the fundamental light on the surface or substrate (e.g., for imaging).
iv) the polarization of the nonlinear light
v) the time-course of i), ii), iii) or iv).
vi) one or more combinations of i), ii), iii), iv) and v).

For example, probe-target binding can be measured by detecting the intensity of nonlinear optical light (e.g., second harmonic light) at some position on a substrate with surface-attached probes; the intensity of the second harmonic light changes as targets bind to the probes at the surface and induce a change in polarization or orientation of indicator molecules. Modeling of the intensity of light with concentration of probe-target binding complexes at the interface can be accomplished using a variety of methods, for instance by calibrating the technique for a given probe-target interaction using radiolabels or fluorescence tags.

The advantages of the present invention are enumerated as follows:
i) Detection of interfacial species in the presence of bulk species in real time. This property can be especially useful when the presence of bulk species are necessary to detect a binding process (eg., if equilibrium or real-time kinetics data is required via, for example, changing target concentrations) or the wash-away step to remove non-bound material is time-consuming, incomplete or gives artifactual results.
ii) Higher signal to noise (lower background) than fluorescence-based detection since SSNLO is generated only at non-centrosymmetric surfaces. SSNLO techniques thus have a very narrow ‘depth of field’. Sources of fluorescence in fluorescence-based detection schemes include that from materials in the field of view but not in the focal plane, autofluorescence, and contamination of the emitted fluorescence with stray excitation light; these are not sources of background nonlinear optical radiation.
iii) The technique is useful when the presence of a liquid solution is required for the measurement, i.e. where the binding process can be obviated or disturbed by a wash-away step. This aspect of the invention can be useful for equilibrium measurements (free energy, binding constants, etc.), which require the presence of bulk species or kinetics measurements with measurements made over a period of time.
iv) The scattering process responsible for the nonlinear effect in SSNLO techniques does not lead to irreversible bleaching of the indicator as quickly as with fluorescent labels; the two-photon absorption cross-section is much lower than the one-photon cross-section in a molecule and the nonlinear technique involves scattering, not absorption.
v) A minimum of collection optics is needed and higher signal to noise is expected since since the fundamental and nonlinear beams (i.e., second harmonic) have well-defined incoming and outgoing directions with respect to the interface. This is advantageous compared to fluorescence-based detection in which the fluorescence is emitted isotropically and there may be a large auto-fluorescence background out of the plane of interest (e.g., the interface containing the probes).
vi) No labels are required, unlike methods with fluorescence-based detection.
vii) Ease of use with beads, cells or other particles whose surface makes an interface with the supporting medium, solution, etc.

### EXAMPLES OF THE USE OF THE INVENTION

Although the present invention may be used in many scientific areas of analysis and, in particular, in the chemical and biological arts, the present invention can be especially useful in genomics or proteomics, where speed and ease of very high-throughput detection are critical. It may be advantageous to detect the surface species in the presence of bulk species—for instance in DNA hybridization or protein-protein detection, the wash-away step for unbound molecules would not be necessary. Moreover in many techniques, such as fluorescence-based detection, a large portion of the sample not at or near the interface (i.e., in the bulk) may contribute undesirably or interfere with measurements. It would be advantageous therefore to use a surface-selective technique such as second harmonic generation or sum frequency generation which is sensitive only to the interface.

SSNLO techniques, when used to study proteins, cells or other molecules in an array format on some surface (two-dimensional ordering of the samples on a solid surface), have other important advantages over the art. Because the technique relies on a scattering (reflection-like) process, the nonlinear beam has a well-defined direction. With fluorescence-based detection the collection optics may be complicated and extensive because the emission is isotropic and only emission from a narrow depth of field is desired. When using nonlinear optical techniques, however, the technique is intrinsically surface-selective—the ‘depth of field’ is confined by the nature of the technique to an extremely thin layer near the interface. Moreoever, the scattered light from the surface possesses a well-defined direction, so that its position at a detector can be mapped directly to a location on the array surface.

Art scanning of microarrays includes confocal-based schemes and non-confocal based schemes. U.S. Pat. No. 5,834,758 (Trulson et al.—relevant portions of which are incorporated by reference herein) describes a non-confocal based scheme for imaging a microarray using fluorescence detection. However, the sample must lie very flat in order to image only within a single focal plane for good out-of-plane discrimination. Therefore, a very finely adjustable translation stage requiring specialized components must be used for this purpose adding to the cost of the instrument and possibly the lifetime as well. The image quality of this type of apparatus can be sensitive to mechanical vibrations. Furthermore, discrimination of the out-of-plane (non-surface bound) fluorophores places a limit on the sensitivity of the technique. U.S. Pat. No. 6,134,002 (Stimson et al.—relevant portions of which are incorporated by reference herein) is an example of a confocal scanning microscope device for imaging a sample plane, i.e. a microarray. Although the confocal-based techniques have good depth discrimination, the scan rate may be low due to descanning requirements and the light throughput can be low, reducing the overall signal to noise ratio and the sensitivity of the technique.

For use with nucleic acid hybridization (oligonucleotide, polynucleotide, RNA, etc.), target oligonucleotides can be reacted with the entire surface; at the probe oligonucleotide sequences in the array (corresponding to known locations) where sequence-complementary hybridization occurs, the fundamental light would give rise to a nonlinear optical signal, or a change in the background of such a signal. This can be detected and correlated with the spatial location of the array element and hence the oligonucleotide sequence.

For example, two major applications of nucleic acid microarrays are: 1) Identification of sequence (gene or gene mutation)—monitoring of DNA variations, for example and 2) Determination of expression level (abundance) of genes. There are many formats for preparing the arrays. For example, in one case probe cDNA (500.about.5000 base pairs long) can be immobilized to a solid surface such as glass using robot spotting and exposed to a set of targets either separately or in a mixture (ref. Ekins and Chu). Another format involves synthesizing oligonucleotides (20.about.25 mer oligos) or peptide nucleic acids probes in-situ (on the solid substrate, Fodor et al.) or by conventional synthesis followed by on-chip immobilization. The array is then exposed to target DNA, hybridized, and the identity or abundance of complementary sequences are determined. Protein arrays can be prepared (see for example, MacBeath and Schreiber, 2000) to determine whether a given target protein binds to the immobilized probe protein on the surface. These arrays were also used to study small molecule binding to the probe proteins. Many reviews of microarray technology and applications are available in the art. For instance, those of: Ramsay (1998—relevant portions of which are incorporated by reference herein), Marshall (1998—relevant portions of which are incorporated by reference herein), Fodor (1997—relevant portions of which are incorporated by reference herein), Duggan et al. (1999—relevant (1998), Marshall (1998), Fodor (1997), Duggan et al. (1999), Schena et al. (1995), Brown et al. (1999), portions of which are incorporated by reference herein), Schena et al. (1995—relevant portions of which are incorporated by reference herein), Brown et al. (1999—relevant portions of which are incorporated by reference herein), McAllister et al. (1997) and Blanchard et al. (1996—relevant portions of which are incorporated by reference herein).

The invention can be used for studying binding processes between other biological components: cells with viruses; protein-protein interactions; protein-ligand; cell-ligand; protein-drugs, nucleic acid-drugs, cell-small molecule; cell-nucleic acid; peptide-cell, oligo or polynucleotides, virus-cell, protein-small molecule, etc. Biomimetic membranes such as phospholipid supported bilayers (eg., egg phosphatidylcholine) can also be used and are particularly useful when studies involve membrane proteins as probes.

Protein arrays can be used to determine whether a given target protein binds to the immobilized probe protein on the surface; these arrays were also used to study small molecule binding to the probe proteins. Protein arrays can be prepared by the method of MacBeath and Schreiber (2000), for example, to determine whether a given target protein binds to the immobilized probe protein on the surface.

The support on which the sequences are formed may be composed from a wide range of material, either biological, nonbiological, organic, inorganic, or a combination of any of these, existing as particles, strands, precipitates, gels, sheets, tubing, spheres, containers, capillaries, pads, slices, films, plates, slides, etc. The substrate may have any convenient shape, such as a disc, square, sphere, circle, etc. The substrate is preferably flat but may take on a variety of alternative surface configurations. For example, the substrate may contain raised or depressed regions on which a sample is located. The substrate and its surface preferably form a rigid support on which the sample can be formed. The substrate and its surface are also chosen to provide appropriate light-absorbing characteristics. For instance, the substrate may be a polymerized Langmuir Blodgett film, functionalized glass, Si, Ge, GaAs, GaP, SiO.sub.2, SiN.sub.4, modified silicon, or any one of a wide variety of gels or polymers such as (poly)tetrafluoroethylene, (poly)vinylidenedifluoride, polystyrene, polycarbonate, or combinations thereof. Other substrate materials will be readily apparent to those of skill in the art upon review of this disclosure. In a preferred embodiment the substrate is flat glass or silica.

According to some embodiments, the surface of the substrate is etched using well known techniques to provide for desired surface features. For example, by way of the formation of trenches, v-grooves, mesa structures, or the like, the synthesis regions may be more closely placed within the focus point of impinging light. The surface may also be provided with reflective “mirror” structures for maximization of emission collected therefrom.

The identity of the probes (e.g., protein structure or oligonucleotide sequence) can vary from site to site across the solid surface, or the same probe can uniformly cover the surface. Targets can be of a single identity or a combination of targets with different identities.

One means of determining whether a particular molecule or particle is a candidate for use as a nonlinear-active indicator is by studying it using second harmonic generation at an air-water interface. For instance, in the case of particles, if the particles assemble at the air-water interface in a manner which gives a net orientation of the particles (on a length scale of the coherence length) the layer of particles will generate second harmonic light. Another means of doing this is by measuring a sample of a suspension of the particles and detecting the hyper-rayleigh scattering. Yet another means involves the use of EFISH (Electric-field induced second harmonic generation). EFISH can be used to determine if a candidate molecule or particle is nonlinearly active. Electric field induced second harmonic (EFISH) is well known in the field of nonlinear optics. This is a third order nonlinear optical effect, with the polarization source written as: P.sup.(2)(.omega..sub.3)=.chi..sup.(2-)(-.omega..sub.3; .omega..sub.1, .omega..sub.2): E.sup..omega. 1 E.sup..omega.2. The effect can be used to measure the hyperpolarizabilty of molecules in solution by using a dc field to induce alignment in the medium, and allowing SHG to be observed. This type of measurement does not require that the particle themselves be ordered at an interface, but does require that the particles be nonlinear active and thus non-centrosymmetric.

The invention can be used for drug screening or high-throughput screening where a candidate drug is tested for its effect on probe-target binding, i.e., to reduce or enhance probe-target binding. In other cases, for example, a drug can be tested for efficacy by its ability to bind to a receptor or other molecule on the surface of a biological cell.

Other examples of the technique's use with arrays include cellular arrays, supported lipid bilayer arrays with or without membrane or attached proteins, etc. Many methods exist in the art for coupling biomolecules (eg., nucleic acid, protein and cells) to solid supports in array format. A wide degree of flexibility may be used in providing the means by which the arrays are created. They can involve, for example, covalent or non-covalent coupling to the substrate or surface directly, to a chemically derivatized substrate, to an intermediate layer of some kind (e.g., self-assembled monolayer, a hydrogel or other bio-compatible layer known in the art). The identity of the probes (e.g., protein structure or oligonucleotide sequence) can vary from site to site across the solid surface, or the same probe can uniformly cover the surface. Targets can be of a single identity or a combination of targets with different identities. The arrays can be prepared in variety of ways including, but not limited to, ink-jet printing, photolithography, micro-contact printing, or any other manner known to one skilled in the art of fabricating them.

Because the binding process can be measured in real time and in the presence of bulk biological components due to the surface-selectivity of the nonlinear optical technique, equilibrium binding curves and kinetics can be measured, the bulk concentration of the components can be varied and a “wash-away” step to remove unbound components, as is used with fluorescence-based detection, may be unnecessary.

In another aspect of the present invention, indicators can be used for imaging studies of cells, membranes and tissues involving techniques such as second harmonic (or sum/difference frequency) microscopy or confocal microscopy. Indicators can be introduced to the sites for imaging in-vivo by well known techniques—e.g., using endoscopy or other well known techniques used in the art for fluorescent-based imaging with dyes. However, indicators may not be required; direct imaging of tissues, etc. in-vivo can be accomplished using a surface-selective nonlinear optical technique and the natural interface created by the tissues or cells in their native environment (e.g., in plasma, in contact with other tissues, in blood, etc.). Imaging tissues in-vitro can also be accomplished in the same manner, with or without the use of indicators.

The present invention can also be used with cells containing ion channel proteins or receptors in the cell membranes (e.g., oocytes with expressed ion channel proteins). Cells can be suspended in some medium, a buffer medium for example. Indicators can also be suspended in the medium. Irradiation of the suspension (in transmission mode) with a fundamental beam leads to generation of the nonlinear optical signal at the cell-medium interface. The ion channel can be of the ligand-gated type. A background nonlinear optical signal is measured at time to; ligand is added to the suspension which serves to modulate the ion channel state or its properties (e.g., opens or closes the ion channel, increases or decreases the ionic permeability of the channel, etc.) which, in turn, leads to a change in the cellular surface electric potential and thus a change in the properties (e.g., intensity) of the nonlinear optical radiation generated near the surface by indicators. The ligand binding to a receptor can be measured quantitatively by following the intensity of nonlinear optical radiation generated as a function of ligand concentration; furthermore, the effect can be monitored in the presence of drug candidates which may, for instance, block or otherwise change the ligand-receptor binding interaction; the binding process can also be measured in real-time to dynamically resolve opening or closing of ion channels. In general, any component can be added to the medium to study its effect on the ligand-receptor interaction using the surface-selective nonlinear optical method described herein.

A wide degree of flexibility is expected in the design of the apparatus including, but not limited to, the source of the fundamental light, the optical train necessary to control, focus, scan or direct the fundamental and nonlinear light beams, the design of the array, the detection system, and the use of a grating or filters and collection optics. The mode of generation (irradiation) or collection can be varied including, for example, the use of evanescent wave (total internal reflection), planar wave guide, reflection or transmission geometries, fiber-optic, near-field illumination, confocal techniques or the use of a microcavity or integrating detection system. A number of art methods for scanning a microarray on a solid surface are described. Examples include U.S. Pat. No.'s Trulson et al. (1998—relevant portions of which are incorporated by reference herein), Trulson et al. (2000), Stern et al. (1997) and Sampas (2000).(2000—relevant portions of which are incorporated by reference herein), Stern et al. (1997—relevant portions of which are incorporated by reference herein) and Sampas (2000—relevant portions of which are incorporated by reference herein).

Because the second harmonic light beam makes a definite angle to the surface plane, one can read-out the properties of the nonlinear optical radiation (for instance, as a function of fundamental incidence position in a two-dimensional array format) without needing to mechanically translate the detector or sample and without extensive collection optics. In the ‘beam scanning’ embodiment, no mechanical translation of sample surface or detector is required—only a change in a direction and/or angle of the fundamental incidence on the sample (for a fixed sample and detector)—the apparatus offers much faster scanning capability, improved ease of manufacturing and a longer lifetime.

The interface can comprise a silica, glass, silicon, polystyrene, nylon, plastic, a metal, semiconductor or insulator surface, or any surface to which biological components can adsorb or be attached. The interface can also include biological cell and liposome surfaces. The attachment or immobilization can occur through a variety of techniques well known in the art. For example, oligonucleotides can be prepared via techniques described in “Microarray Biochip Technology”, M. Schena (Ed.), Eaton Publishing, 1998—relevant portions of which are incorporated by reference herein. And, for example with proteins, the surface can be derivatized with aldehyde silanes for coupling to amines on surfaces of biomolecules (MacBeath and Schreiber, 2000, relevant portions of which are incorporated by reference herein). BSA-NHS (BSA-N-hydroxysuccinimide) surfaces can also be used by first attaching a molecular layer of BSA to the surface and then activating it with N,N′-disuccinimidyl carbonate. The activated lysine, aspartate or glutamate residues on the BSA react with surface amines on the proteins.

Supported phospholipid bilayers can also be used, with or without membrane proteins or other membrane-associated components as, for example, in Salafsky et al., Biochemistry, 1996—relevant portions of which are incorporated by reference herein, “Biomembranes”, Gennis, Springer-Verlag, Kalb et al., 1992 and Brian et al., 1984. Supported phospholipid bilayers are well known in the art and there are numerous techniques available for their fabrication, with or without associated membrane proteins. These supported bilayers typically must be submerged in aqueous solution to prevent their destruction when they become exposed to air.

If a solid surface is used (e.g., planar substrate, beads, etc.) it can also be derivatized via various chemical reactions to either reduce or enhance its net surface charge density to optimize the detection of probe-target interactions (e.g., a hybridization process).

The binding process can be performed in the presence of small molecules, drugs, blocking agents, or other components which modulate the binding process.

The surface arrays can be constructed according a plurality of methods found in the art. For DNA microarrays, most are prepared with one of three non-standard approaches (Case-Green, 1998): Affymetrix, Inc. probe arrays are prepared using patterned, light-directed combinatorial chemical synthesis (Fodor, 1997); spotted arrays can be made according to Duggan (1999), Schena (1995), Brown and Botstein (1999) and McAllister (1997); ink-jet techniques can also be used to synthesize oligonucleotides base by base through sequential solution-based reactions on an appropriate substrate (Blanchard, 1996—relevant portions of which are incorporated by reference herein).

For example, nucleic acid, oligo- or nucleotide arrays can be constructed according to U.S. Pat. Nos. 6,110,426, 5,143,854, 6,110,426—relevant portions of which are incorporated by reference herein, U.S. Pat. No. 5,143,854—relevant portions of which are incorporated by reference herein or Fodor (1991). Soluble protein arrays can be constructed according to Ekins (1999) relevant portions of which are incorporated by reference herein. Membrane protein arrays can be constucted by micropatterning of fluid lipid membranes according, for example, to the method of Groves et al. (1997—relevant portions of which are incorporated by reference herein). The array substrate can be composed of glass, silicon, indium tin oxide, or any other substrate known in the art. The surface array under study can contain physical barriers between elements so that the elements (and their biomolecules) can remain in isolation from each other during a chemical reaction step. The array locations can consist of different probes, the same probes everywhere, or some combination thereof. The array can also be constructed on the underside of a prism allowing for total internal reflection of the beam and evanescent generation of the nonlinear light. Or an array substrate can be brought into contact with a prism with the same result.

An electrophoretic system can also be used in conjunction with the surface array, for example to provide reagents or biological components to one or a plurality of locations using flow channels or microcapillaries. For instance, the sample can include an array of microcapillary channels, each distinct from the other and each allowing a target-probe reaction to occur; the imaging technique would then consist of array elements, each one a microcapillary channel or reaction chamber into which the channel feeds and drains.

Examples of samples in which indicators can be of use include, but are not limited to, solid surfaces with immobilized protein, oligonucleotides or cells and supported phospholipid bilayers. The surface geometry can be varied, indeed spherical beads and other non-planar geometries are generally accessible with the nonlinear optical techniques.

The polarization of the fundamental and nonlinear beams can be selected with polarizing optics elements. By analyzing the intensity of the nonlinear beam as a function of fundamental and nonlinear polarization, more information (e.g., higher signal to noise) about the probe-target complexes can be obtained. Furthermore, by selecting and analyzing the polarization of the fundamental or nonlinear optical radiation, background radiation can be reduced.

Detection can be accomplished with the use of multiple internal reflection plates (N.J. Harrick—relevant portions of which are incorporated by reference herein) allowing the fundamental beam to make multiple contacts with the array surface, thus increasing the intensity of the generated nonlinear light. Another alternative is to construct an optical cavity with the array surface on one side and a lossy coupler at one end to permit the output coupling of the nonlinear light, creating an optical microcavity which would allow the buildup of very high intensities under resonance and thus increase the amount of nonlinear light generated.

Cells bound to a substrate can be used to determine protein-cell binding, virus-cell binding, etc. where the cell is the probe component and proteins, viruses, etc. are the target components. The next section discusses the well known art for coupling cells to solid substrates. Various art not involving the use of a surface-selective nonlinear optical technique contains relevant portions for the present invention and the following exemplary list and their references therein is referenced herein: King et al., U.S. Pat. No. 5,633,724 for the scanning and analysis of the scans; Fork et al., U.S. Pat. No. 6,121,983 for the multiplexing of a laser to produce a laser array suitable for scanning; Foster, U.S. Pat. No. 5,485,277; Fodor et al., U.S. Pat. No. 5,324,633 and Fodor et al., U.S. Pat. No. 6,124,102 for a substrate containing an array of attached probes and for the analysis of scans to determine kinetic and equilibrium properties of a binding reaction between probes and targets; Kain et al., U.S. Pat. No. 5,847,400 for laser scanning of a substrate; King et al., U.S. Pat. No. 5,432,610 for an optical resonance cavity for power build-up; Walt et al., U.S. Pat. No. 5,320,814, Walt et al., U.S. Pat. No. 5,250,264, Walt et al., U.S. Pat. No. 5,298,741, Walt et al., U.S. Pat. No. 5,252,494, Walt et al., U.S. Pat. No. 6,023,540, Walt et al., U.S. Pat. No. 5,814,524, Walt et al., U.S. Pat. No. 5,244,813 for fiber-optic-based apparatus; Fiekowsky et al., U.S. Pat. No. 6,095,555 for imaging and software-based analysis of images; Stern et al., U.S. Pat. No. 5,631,734 for data acquisition; Stimson et al., U.S. Pat. No. 6,134,002 for confocal imaging techniques; Sampas, U.S. Pat. No. 6,084,991 for CCD-based imaging techniques; Stern et al., U.S. Pat. No. 5,631,734 for photolithographical preparation of probes attached to surfaces; Shalon et al., U.S. Pat. No. 6,110,426 for methods and apparatus for creating attached probes on a surface; Slettnes, U.S. Pat. No. 6,040,586 for position-based scanning techniques; Trulson et al, U.S. Pat. No. 6,025,601 for methods of imaging probe-target binding on a surface.

Microarrays of Cells

This section outlines some of the methods concerned with fabricating arrays of biological cells on surfaces, one type of array amenable to study using the present invention. Many methods have been described for making uniform micro-patterned arrays of cells for other applications, using for example photochemical resist-photolithograpy. (Mrksich and Whitesides, Ann. Rev. Biophys. Biomol. Struct. 25:55-78, 1996—relevant portions of which are incorporated by reference herein). According to this photoresist method, a glass plate is uniformly coated with a photoresist and a photo mask is placed over the photoresist coating to define the “array” or pattern desired. Upon exposure to light, the photoresist in the unmasked areas is removed. The entire photolithographically defined surface is uniformly coated with a hydrophobic substance such as an organosilane that binds both to the areas of exposed glass and the areas covered with the photoresist. The photoresist is then stripped from the glass surface, exposing an array of spots of exposed glass. The glass plate then is washed with an organosilane having terminal hydrophilic groups or chemically reactable groups such as amino groups. The hydrophobic organosilane binds to the spots of exposed glass with the resulting glass plate having an array of hydrophilic or reactable spots (located in the areas of the original photoresist) across a hydrophobic surface. The array of spots of hydrophilic groups provides a substrate for non-specific and non-covalent binding of certain cells, including those of neuronal origin (Klienfeld et al., J. Neurosci. 8:4098-4120, 1988—relevant portions of which are incorporated by reference herein). Reactive ion etching has been similarly used on the surface of silicon wafers to produce surfaces patterned with two different types of texture (Craighead et al., Appl. Phys. Lett. 37:653, 1980—relevant portions of which are incorporated by reference herein; Craighead et al., J. Vac. Sci. Technol. 20:316, 1982—relevant portions of which are incorporated by reference herein; Suh et al. Proc. SPIE 382:199, 1983—relevant portions of which are incorporated by reference herein).

In another method based on specific yet non-covalent interactions, photoresist stamping is used to produce a gold surface coated with protein-adsorptive alkanethiol. (Singhvi et al., Science 264:696-698, 1994—relevant portions of which are incorporated by reference herein). The bare gold surface is then coated with polyethylene-terminated alkanethiols that resist protein adsorption. After exposure of the entire surface to laminin, a cell-binding protein found in the extracellular matrix, living hepatocytes attach uniformly to, and grow upon, the laminin coated islands (Singhvi et al. 1994).

An elaboration involving strong, but non-covalent, metal chelation has been used to coat gold surfaces with patterns of specific proteins (Sigal et al., Anal. Chem. 68:490-497, 1996—relevant portions of which are incorporated by reference herein). In this case, the gold surface is patterned with alkanethiols terminated with nitriloacetic acid. Bare regions of gold are coated with tri(ethyleneglycol) to reduce protein adsorption. After adding Ni.sup.2+, the specific adsorption of five histidine-tagged proteins is found to be kinetically stable.

More specific uniform cell-binding can be achieved by chemically crosslinking specific molecules, such as proteins, to reactable sites on the patterned substrate. (Aplin and Hughes, Analyt. Biochem. 113:144-148, 1981—relevant portions of which are incorporated by reference herein). Another elaboration of substrate patterning optically creates an array of reactable spots. A glass plate is washed with an organosilane that chemisorbs to the glass to coat the glass. The organosilane coating is irradiated by deep UV light through an optical mask that defines a pattern of an array. The irradiation cleaves the Si—C bond to form a reactive Si radical. Reaction with water causes the Si radicals to form polar silanol groups. The polar silanol groups constitute spots on the array and are further modified to couple other reactable molecules to the spots, as disclosed in U.S. Pat. No. 5,324,591—relevant portions of which are incorporated by reference herein, incorporated by reference herein. For example, a silane containing a biologically functional group such as a free amino moiety can be reacted with the silanol groups. The free amino groups can then be used as sites of covalent attachment for biomolecules such as proteins, nucleic acids, carbohydrates, and lipids. Other methods for patterning the adhesion of mammalian cells to surfaces using self-assembled monolayers on a surface include Lopez et al. 1993 and Stenger et al., 1992—relevant portions of which are incorporated by reference herein.

The non-patterned covalent attachment of a lectin, known to interact with the surface of cells, to a glass substrate through reactive amino groups has been demonstrated (Aplin & Hughes, 1981). The optical method of forming a uniform array of cells on a support requires fewer steps and is faster than the photoresist method, (i.e., only two steps), but it requires the use of high intensity ultraviolet light from an expensive light source.

In all of these methods the resulting array of cells is uniform, since the biochemically specific molecules are bound to the micro-patterned chemical array uniformly. In the photoresist method, cells bind to the array of hydrophilic spots and/or specific molecules attached to the spots which, in turn, bind cells. Thus cells bind to all spots in the array in the same manner. In the optical method, cells bind to the array of spots of free amino groups by adhesion. Methods for attaching a variety of cell types to the same substrate for simultaneously binding against these cell types also exists.

Peptide-Nucleic Acids

In an alternative embodiment, peptide nucleic acids or oligomers, which are analogs of nucleic acids in which, for example, the peptide-like backbone is replaced with an uncharged backbone, can be used with the present invention. PNAs are well known in the art. References below give extensive reviews of the use of these nucleic acid analogs in a wide range of applications, including surface and array-based hybridization wherein PNAs are attached to surfaces and allowed to bind with sequence-complementary DNAs or RNAs.

For instance, oligomers of PNA can be used as the surface-attached probe components instead of DNA oligomers. A key advantage to using PNAs is that the hybridization reaction with DNAs or RNAs, for example, (containing charged phosphate groups) is only weakly dependent (eg., the melting temperature) on ionic strength because there is much less charge repulsion as found with conventional DNA-DNA, etc. hybridization. Thus, one can use the surface-selective nonlinear optical technique to follow a probe-target hybridization at any desired ionic strength—in order to maximize the polarization response of the indicators oriented by the charged surface. For example, if PNA probes are attached to the surface and allowed to react with DNA targets, the hybridization will result in a net increase in negative charge on the surface, thus changing the amount of indicators polarized by the interface and, in turn, the amount of nonlinear optical light (eg., second harmonic light) generated.

One aspect of the advantage of not having a strong ionic dependence is that low ionic strengths can be used, thus increasing the Debye length of the system and increasing the distance from the surface at which indicators become polarized; this can reduce the contribution of the orientation of the PNAs to the polarization of the water—i.e., if PNAs are randomly oriented on the surface, the polarization response near to the surface can approach zero, but indicators much farther from the surface can be polarized since the orientation effect is diminished far from the surface.

The PNAs are commercially available (Applied Biosystems, Foster City, Calif.) or other analogs of DNA can be synthesized and used.

The following references are broad reviews of the use of PNAs.
Nielsen, et al. “Peptide nucleic acids (PNA): Oligonucleotide analogues with a polyamide backbone” Antisense Research and Applications (1992) 363-372
Nielsen, et al. “Peptide nucleic acids (PNAs): Potential Antisense and Anti-gene Agents.” Anti-Cancer Drug Design 8 (1993) 53-63
Buchardt, et al. “Peptide nucleic acids and their potential applications in biotechnology” TIBTECH 11 (1993) 384-386
Nielsen, P. E., Egholm, M. and Buchardt, 0. “Peptide Nucleic Acid (PNA). A DNA mimic with a peptide backbone” Bioconjugate Chemistry 5 (1994) 3-7
Nielsen “Peptide nucleic acid (PNA): A lead for gene therapeutic drugs” Antisense Therapeutics 4 (1996) 76-84
Nielsen, P. E. “DNA analogues with nonphosphodiester backbones” Annu. Rev. Biophys. Biomol. Struct. 24 (1995) 167-183
Hyrup, B. and Nielsen, P. E. “Peptide Nucleic Acids (PNA): Synthesis, Properties and Potential Applications” Bioorg. Med. 4 (1996) 5-23
Mesmaeker, A. D., Altman, K.-H., Waldner, A. and Wendebom, S. “Backbone modifications in oligonucleotides and peptide nucleic acid systems” Curr. Opin. Struct. Biol. 5 (1995) 343-355
Noble, et al. “Impact on Biophysical Parameters on the Biological Assessment of Peptide Nucleic Acids, Antisense Inhibitors of Gene Expression” Drug.Develop.Res. 34 (1995) 184-195
Dueholm, K. L. and Nielsen, P. E. “Chemistry, properties, and applications of PNA (Peptide Nucleic Acid)” New J. Chem. 21 (1997) 19-31
Knudsen and Nielsen “Application of Peptide Nucleic Acid in Cancer Therapy” Anti-Cancer Drug 8 (1997) 113-118
Nielsen, P. E. “Design of Sequence-Specific DNA-Binding Ligands” Chem. Eur. J. 3 (1997) 505-508
Corey “Peptide nucleic acids: expanding the scope of nucleic acid recognition” TIBTECH 15 (1997) 224-229
Nielsen, P. E. and 0.0 slashed.rum, H. “Peptide nucleic acid (PNA), a new molecular tool.” In Molecular Biology: Current Innovations and Future Trends, Part2. Horizon Scientific Press, (1995) 73-89
Nielsen, P. E. and Haaima, G. “Peptide nucleic acid (PNA). A DNA mimic with a pseudopeptide backbone” Chem. Soc. Rev. (1997) 73-78
.O slashed.rum, H., Kessler, C. and Koch, T. “Peptide Nucleic Acid” Nucleic Acid Amplification Technologies: Application to Disease Diagnostics (1997) 29-48
Wittung, P., Nielsen, P. and Norden, B. “Recognition of double-stranded DNA by peptide nucleic acid” Nucleosid. Nucleotid. 16 (1997) 599-602
Weisz, K. “Polyamides as artificial regulators of gene expression” Angew. Chem. Int. Ed. Eng 36 (1997) 2592-2594
Nielsen, P. E. “Structural and Biological Properties of Peptide Nucleic Acid (Pna)” Pure & Applied Chemistry 70 (1998) 105-110
Nielsen, P. E. “Sequence-specific recognition of double-stranded DNA by peptide nucleic acids” Advances in DNA Sequence-Specific Agents 3 (1998) 267-278
Nielsen “Antisense Properties of Peptide Nucleic Acid” Handbook of Experimental Pharmacology 131 (1998) 545-560
Nielsen “Peptide Nucleic Acids” Science and Medicine (1998) 48-55
Uhlmann, E. “Peptide nucleic acids (PNA) and PNA-DNA chimeras: from high binding affinity towards biological function” Biol Chem 379 (1998) 1045-52
Wang “DNA biosensors based on peptide nucleic acid (PNA) recognition layers. A review” Biosens Bioelectron 13 (1998) 757-62
Uhlmann, E., Peyman, A., Breipohl, G. and Will, D. W. “PNA: Synthetic polyamide nucleic acids with unusual binding properties” Angewandte Chemie-International Edition 37 (1998) 2797-2823
Nielsen, P. E. “Applications of peptide nucleic acids” Curr Opin Biotechnol 10 (1999) 71-75
Bakhtiar, R. “Peptide nucleic acids: deoxyribonucleic acid mimics with a peptide backbone” Biochem. Educ. 26 (1998) 277-280
Lazurkin, Y. S. “Stability and specificity of triplexes formed by peptide nucleic acid with DNA” Mol. Biol. 33 (1999) 79-83
Nielsen and Egholm “Peptide Nucleic Acids: Protocols and Applications” (1999) 266 pp.
Eldrup and Nielsen “Peptide nucleic acids: potential as antisense and antigene drugs” Adv. Amino Acid Mimetics Peptidomimetics 2 (1999) 221-245
Bentin, T. and Nielsen, P. E. “Triplexes involving PNA” Triple Helix Form. Oligonucleotides (1999) 245-255
Falkiewicz, B. “Peptide nucleic acids and their structural modifications” Acta Biochim. Pol. 46 (1999) 509-529.

The following references are descriptions of the use of PNAs in array-based detection, including means for attaching the PNA probes to the solid surface.
Hoffmann, R., et al. “Low scale multiple array synthesis and DNA hybridization of peptide nucleic acids” Pept. Proc. Am. Pept. Symp., 15th (1999) 233-234
Matysiak, S., Hauser, N.C., Wurtz, S. and Hoheisel, J. D. “Improved solid supports and spacer/linker systems for the synthesis of spatially addressable PNA-libraries” Nucleosides Nucleotides 18 (1999) 1289-1291.

Definitions

The following terms used throughout the present specification are intended to have the following general definitions:

Definitions

The following terms used throughout the present specification are intended to have the following general definitions:

1. Complementary: Refers to the topological and chemical compatibility of interacting surfaces between two biological components, such as with a ligand molecule and its receptor (also referred to sometimes in the art as ‘molecular recognition’). Thus, the receptor and its ligand can be described as complementary, and, furthermore, the contacts' surface characteristics are complementary to each other.

2. Biological (Components): This term includes any naturally occurring or modified particles or molecules found in biology, or those molecules and particles which are employed in a biological study, including probes and targets. Examples of these include, but are not limited to, a biological cell, protein, nucleic acids, antibodies, receptors, peptides, small molecules, oligonucleotides, carbohydrates, lipids, liposomes, polynucleotides and others such as drugs, toxins and genetically engineered protein or peptide.

3. Ligand: A ligand is a molecule that is recognized by a particular receptor. Examples of ligands that can be used with the present invention include, but are not restricted to, antagonists or agonists for cell membrane receptors, toxins and venoms, viral epitopes, hormones, hormone receptors, peptides, enzymes, enzyme substrates, cofactors, drugs (e.g. opiates, steroides, etc.), lectins, sugars, oligonucleotides, nucleic acids, oligosaccharides, proteins, and monoclonal antibodies.

4. Receptor: A molecule that has a chemical affinity for a given ligand. Receptors can be naturally occurring or man-made molecules. Also, they can be used in an unaltered state or as aggregates with other biological components. Receptors can be attached, covalently or noncovalently, to a binding partner, either directly or via a specific binding substance. Examples of receptors which can be employed by this invention include, but are not limited to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes and organelles. Receptors are occasionally referred to in the art as anti-ligand. As the term receptors is used herein, no difference in meaning is intended. A “Ligand Receptor Pair” is formed when two macromolecules have combined through molecular recognition to form a complex.

Other examples of receptors which can be investigated by this invention include but are not restricted to:

a) Microorganism receptors: Determination of ligands which bind to receptors, such as specific transport proteins or enzymes essential to survival of microorganisms, is useful in developing a new class of antibiotics. Of particular value would be antibiotics against opportunistic fungi, protozoa, and those bacteria resistant to the antibiotics in current use.

b) Enzymes: For instance, one type of receptor is the binding site of enzymes such as the enzymes responsible for cleaving neurotransmitters; determination of ligands which bind to certain receptors to modulate the action of the enzymes which cleave the different neurotransmifters is useful in the development of drugs which can be used in the treatment of disorders of neurotransmission.

c) Antibodies: For instance, the invention can be useful in investigating the ligand-binding site on the antibody molecule which combines with the epitope of an antigen of interest; determining a sequence that mimics an antigenic epitope can lead to the development of vaccines of which the immunogen is based on one or more of such sequences or lead to the development of related diagnostic agents or compounds useful in therapeutic treatments such as for autoimmune diseases (e.g., by blocking the binding of the “self” antibodies).

d) Nucleic Acids: Sequences of nucleic acids can be synthesized to establish DNA or RNA binding sequences.

e) Catalytic Polypeptides: Polymers, preferably polypeptides, which are capable of promoting a chemical reaction involving the conversion of one or more reactants to one or more products. Such polypeptides generally include a binding site specific for at least one reactant or reaction intermediate and an active functionality proximate to the binding site, which functionality is capable of chemically modifying the bound reactant. Catalytic polypeptides are described in, for example, U.S. Pat. No. 5,215,899, which is incorporated herein by reference for all purposes.

f) Hormone receptors: Examples of hormone receptors include, e.g., the receptors for insulin and growth hormone. Determination of the ligands which bind with high affinity to a receptor is useful in the development of, for example, an oral replacement of the daily injections which diabetics must take to relieve the symptoms of diabetes, and in the other case, a replacement for the scarce human growth hormone which can only be obtained from cadavers or by recombinant DNA technology. Other examples are the vasoconstrictive hormone receptors; determination of those ligands which bind to a receptor can lead to the development of drugs to control blood pressure.

g) Opiate receptors: Determination of ligands which bind to the opiate receptors in the brain is useful in the development of less-addictive replacements for morphine and related drugs.

h) Ion channel proteins or receptors, or cells containing ion channel receptors.

5. Surface-selective: This term refers to a non-linear optical technique such as second harmonic generation or sum/difference frequency generation in which, by symmetry, only a non-centrosymmetric surface (comprising array, substrate, solution, biological components, etc.), is capable of generating non-linear light.

6. Array: Refers to a substrate or solid support on which is fabricated one type, or a plurality of types, of biological components in one or a plurality of known locations. This includes, but is not resticted to, two-dimensional microarrays and other patterned samples. Other terms in the art which are often used interchangeably for ‘array’ include: gene chip, gene array, biochip, DNA chip, protein chip and microarray, the latter being an array with elements of the array (patterned areas with attached probes) whose dimensions are on the order of microns.

7. Label: Refers to a nonlinear-active moiety, particle or molecule which can be attached (covalently or non-covalently) to a molecule, particle or phase (e.g., lipid bilayer) in order to render the latter more nonlinear optical active.

8. Linker: A molecule which serves to chemically link (usually via covalent bonds) two different objects together. Herein a linker can be used to couple targets to non-linear active particles or moieties, targets to nonlinear-active derivatized particles, surface layers to targets, surface layers to nonlinear-active particle or moieties, etc. A linker can, for example, be a homobifunctional or heterobifunctional cross-linker molecule, a biotin-streptavidin couple wherein the biotin is attached to one of the two objects and the streptavidin to the other, etc. Many linkers are available commercially, for example from Pierce Chemical Inc., Sigma-Aldrich, Fluka, etc. In some art, the term ‘tether’, ‘spacer’ or ‘cross-linker’ is also used with the same meaning.

9. Elements: When used with ‘array’ or ‘microarray’, the meaning is a specific location among the plurality of locations on the array surface. Each element is a discrete region of finite area formed on the surface of a solid support or substrate.

10. Nonlinear: Refers herein to those optical techniques capable of transforming the frequency of an incident light beam (called the fundamental). The nonlinear beams are the higher order frequency beams which result from such a transformation, e.g. second harmonic, etc. In second harmonic, sum frequency or difference frequency generation, the nonlinear beams are generated coherently. In second harmonic generation (SHG), two photons of the fundamental beam are virtually scattered by the interface to produce one photon of the second harmonic. Also referred to herein as nonlinear optical or surface-selective nonlinear (optical) or by various combinations thereof.

11. Probe: Refers herein to biological components (eg., cells, proteins, virus, ligand, small molecule, drugs, oligonucleotides, DNA, RNA, cDNA, etc.) which are attached to a surface (e.g., solid substrate, cell surface, liposome surface, etc.), or are cells, lipsomes, particles, beads or other components which comprise a surface e.g. freely suspended in some medium in a sample cell. (In some literature in the art, this term refers to the free components which are tested for binding against the probes).
12. Target: Refers herein to biological components which are unbound to the probes' surface or surfaces comprising attached probes, and which may bind to probes.

13. Attached (Attach): Refers herein to biological components which are either prepared or engineered in-vitro to be attached to some surface, via covalent or non-covalent means, including for example the use of linker molecules to, for example, a solid substrate, a cell surface, a liposome surface, a gel substrate, etc.; or the probes are found naturally ‘attached’ to a surface such as in the example of native membrane receptors embedded in cell membranes, tissues, organs (in-vitro or in-vivo). In some instances herein, the word ‘attached’ or ‘attach’ refers also to the chemical or physical attachment of a linker or spacer to a target. Also referred to herein as ‘surface-attached’.

14. Centrosymmetric: A molecule or material phase is centrosymmetric if there exists a point in space (the ‘center’ or ‘inversion center’) through which an inversion (x,y,z).fwdarw.(-x,-y,-z) of all atoms is performed that leaves the molecule or material unchanged. A non-centrosymmetric molecule or material lacks this center of inversion. For example, if the molecule is of uniform composition and spherical or cubic in shape, it is centrosymmetric. Centrosymmetric molecules or materials have no nonlinear susceptibility or hyperpolarizability, necessary for second harmonic, sum frequency and difference frequency generation.

15. Nucleic Acid Analog: A non-natural nucleic acid which can function as a natural nucleic acid in some way. For example, a Peptide Nucleic Acid (PNA) is a non-natural nucleic acid because it has a peptide-like backbone rather than the phosphate background of natural nucleic acids. The PNAs can hybridize to natural nucleic acids via base-pair interactions. Another example of a Nucleic acid analog can be one in which the base pairs are non-natural in some way.

16. Indicator: Refers to a nonlinear active molecule or particle (possesses a hyperpolarizability) whose nonlinear optical properties or orientation near a surface or interface is modulated as the electric charge density or potential of the surface is modulated. In one aspect of the invention, the charge or potential of an interface is modulated by the binding of a target to probes immobilized on the surface. In another aspect, the surface electric potential of a cell is changed by a change in the ion channel properties—an opening, closing, increase or decrease in ionic permeability in response to target (ligand) binding, for instance. In another aspect of the invention, an indicator serves as a marker for imaging purposes, e.g., to image cells or tissues. An indicator does not appreciably alter or participate in the target-probe reaction itself. The indicator is dissolved or suspended in the liquid, medium, solution or aqueous phase containing the target component. An indicator as defined herein does not translocate appreciably into the lipid bilayer of vesicles or cells. An indicator must possess freedom of movement to respond to changes in surface electric charge density or potential. Measuring the nonlinear optical response of a glass-solvent or glass-water interface, in the presence of dissolved or suspended indicators in the water or solvent, would be one means of assaying whether a candidate molecule or indicator would function as an indicator: because glass carries a net negative charge, if the intensity of the nonlinear optical radiation generated by the interface in the presence of the molecule is greater than the background without it, the molecule could function as an indicator. Another means of assaying for a candidate molecule's ability as an indicator is by measuring the intensity of nonlinear optical radiation generated by a semiconductor-liquid interface as a function of applied voltage (and hence surface electric charge density) between the semiconductor and the bulk of the liquid. Yet another means is to measure the hyper-rayleigh scattering (HRS) from a solution or suspension of the indicator candidates; if HRS is generated and the candidate itself is charged or dipolar, it may serve well as an indicator.

17. Binding Affinity or Affinity: The specific physico-chemical interactions between binding partners, such as a probe and target, which lead to a binding complex (affinity) between them. The binding reaction is characterized by an equilibrium constant which is a measure of the energetic strength of binding between the partners. Specificity in a binding reaction implies that probe-target binding only occurs appreciably with specific binding partners—not any at random. For example, the protein Immunoglobulin G (IgG) has a specific binding affinity for protein G and less or none for other proteins. In some art, the term ‘molecular recognition’ is used to describe the binding affinity between components.

18. Electrically Charged or Electric Charge: Defined herein as net electric charge on a particle or molecule, which confers a mobility (velocity) of said particle or molecule in an electric field. The net charge could be part of a molecular moiety such as phosphate group on nucleic acid backbones, side-chains of amino acid residues in proteins, lipid head groups in membrane lipids or cellular membranes, etc. The charge can be positive or negative and would determine the direction of mobility of the particle or molecule if said particle or molecule is placed in an electric field of a given orientation (direction of positive to negative electric potential). The charge can be non-integer multiples of the fundamental unit of charge (q.apprxeq. 1.6.times. 10.sup.-19 C) or a fraction of the fundamental unit of charge—so-called ‘partial charges’, well known to those skilled in the art.

19. Dipolar: Defined herein as possessing an electric dipole or ‘dipole moment’ on a particle or molecule, which takes the standard definition known to one skilled in the art: the sum of all vectors .mu.=Q.multidot.R where Q is the amount of charge (positive or negative) at a particular spatial location (x,y,z in Cartesian coordinates) in the particle or molecule and R is the vector which points from an origin of reference (x,y,z) to the net charge Q. If the sum of these vectors results in a vector with a non-zero trace (sum of x,y,z components of the resultant vector), the particle or molecule possesses a dipole moment and is electrically dipolar.

20. Electrically Neutral: Defined herein as zero net (sum of positive and negative) electric charge on a particle or molecule, which would result in no appreciable mobility (velocity) of said particle or molecule in an electric field.

21. Hyperpolarizability or Nonlinear Susceptibility: The properties of a molecule, particle, interface or phase which allow for generation of the nonlinear light. Typical equations describing the nonlinear interaction for second harmonic generation are:
.alpha..sup.(2)(2.omega.)-=.beta.:E(.omega.).multidot.E(.omega.) or
P.sup.(2)(2.omega.)=.chi..sup.(2-):E(.omega.))E(.omega.) where alpha. and P are, respectively,
the induced molecular and macroscopic dipoles oscillating at frequency 2.omega., .beta. and .chi..sup.(2) are, respectively, the hyperpolarizability and second-harmonic (nonlinear) susceptibility tensors, and E(.omega.) is the electric field component of the incident radiation oscillating at frequency omega. The macroscopic nonlinear susceptibility .chi..sup.(2) is related by an orientational average of the microscopic beta. hyperpolarizability. For sum or difference frequency generation, the driving electric fields (fundamentals) oscillate at different frequencies (i.e., omega..sub. 1 and omega..sub.2) and the nonlinear radiation oscillates at the sum or difference frequency (.omega..sub. 1.+-..omega..s-ub.2). The terms hyperpolarizability, second-order nonlinear polarizability and nonlinear susceptibility are sometimes used interchangeably, although the latter term generally refers to the macroscopic nonlinear-activity of a material or chemical phase or interface. The terms ‘nonlinear active’ or ‘nonlinearly active’ used herein also refer to the general property of the ability of molecules, particles, an interface or a phase, to generate nonlinear optical radiation when driven by incident radiation beam or beams.

22. Polarization: The net dipole per unit volume (or area) in a region of space. The polarization can be time-dependent or stationary. Polarization is defined as: .intg..mu.(R) dR where an integration of the net dipole is made over all volume elements in space dR near an interface.

23. Radiation: Refers herein to electromagnetic radiation or light, including the fundamental beams used to generate the nonlinear optical effect, or the nonlinear optical beams which are generated by the fundamental. Also referred to herein as ‘waves’ or ‘nonlinear signal’ or ‘signal’, ‘beams’ or ‘light’.

24. Near-field techniques: Those techniques known in the art to be capable of measuring or imaging optical radiation on a surface or substrate with a lateral resolution at or smaller than the diffraction-limited distance. Examples of near-field techniques (or near-field imaging) include NSOM (near-field scanning optical microscopy), whereby optical radiation (from fluorescence, second harmonic generation, etc.) is collected at a point very near the surface.

25. Detecting, Detection: When referring herein to nonlinear optical methods, refers to those techniques by which the properties of surface-selective nonlinear optical radiation can be used to detect, measure or correlate properties of probe-target interactions, or effects of the interactions, with properties of the nonlinear optical light (e.g., intensity, wavelength, polarization or other property common to electromagnetic radiation).

26. Interface: For the purpose of this invention, the interface can be defined as a region which generates a nonlinear optical signal or the region near a surface in which there are nonlinear-active labeled targets possessing a net orientation. An interface can also be composed of two surfaces, a surface in contact with a different medium (e.g., a glass surface in contact with an aqueous solution, a cell surface in contact with a buffer), the region near the contact between two media of different physical or chemical properties, etc.

27. Conjugated, Coupled: Refers herein to the state in which one particle, moiety or molecule is chemically bonded, covalently or non-covalently linked or by some means attached to a second particle moiety, molecule, surface or substrate. These means of attachment can be via electrostatic forces, covalent bonds, non-covalent bonds, physisorption, chemisorption, hydrogen bonds, van der Waal's forces or any other force which holds the probes with a binding energy to the substrate (a corallery to this definition is that some force is required to separate the probes held by the substrate from the substrate).

28. Reactions: Refers herein to chemical, physical or biological reactions including, but not limited to, the following: probes, targets, inhibitors, small molecules, drugs, antagonists, antibodies, etc. The term ‘effects of reactions’ or ‘effects of said reactions’ refers herein to physical or chemical effects of the probe-target reactions: for example, the probe-target reactions can comprise a ligand-receptor binding reaction which leads, in turn, to an ion channel opening and a change in the surface charge density of a cell, the latter being then detected by the nonlinear optical technique. The effects of the probe-target reactions, or the probe-target reactions themselves, might be referred in some art as a ‘second messenger’ reaction. In some cases, reactions' and ‘interactions’ are used interchangeably.

29. Surface layer: Refers herein to a chemical layer which functionally derivatives the surface of a solid support. For instance, the surface chemical groups can be changed by the derivatization layer according to the particular chemical functionality of the derivatizing agent. For instance, a silica bead with negatively charged silanol groups on its surface can be converted to an amine-reactive, amine-containing, etc. surface via organosilane reagents.

30. Delivery, Illumination, Collection: In the context of manipulation of optical radiation (e.g., light beams), delivery and illumination refer herein to the guiding of the fundamental beam to the interface or regions of interest at an interface; collection refers to the optical collection of the nonlinear light produced at the interface (e.g., second harmonic light).

31. Inhibitor, inhibiting: Defined herein as moieties, molecules, compounds or particles which bind to probes in competition with targets; the probe-target interactions are decreased or prevented in the presence of an inhibitor compound, molecule or particle. Blocking agents refers herein to those compounds, molecules, moieties or particles which prevent probe-target interactions (e.g., binding reactions between probes and targets).

32. Agonist: Defined herein as moieties, molecules, compounds or particles which activate an intracellular response when they bind to a receptor.

33. Antagonist: Defined herein as moieties, molecules, compounds or particles which competitively bind to a receptor on a cell surface at the same site as agonists, but which do not activate the intracellular response initiatied by the active form of the receptor (e.g., activated by agonist binding), and can thereby inhibit the intracellular responses of agonists or partial agonists. Antagonists do not diminish the baseline intracellular response in the absence of an agonist or partial agaonist.

34. Partial Agonist: Defined herein as moieties, molecules, compounds or particles which activate the intracellular response when they bind to a receptor on the cell surface to a lesser degree or extent than do agonists.

35. Interactions: Defined herein as some physical or chemical reaction or interaction between components in a sample. For example, the interactions can be physico-chemical binding reactions between a probe and a target, dipole-dipole attraction or repulsion between two molecules, van der Waals interactions between two atomic or molecular species, a chemical affinity interaction, a covalent bond between molecules, a non-covalent bond between molecules, an electrostatic interaction (repulsive or attractive), a hydrogen bond and others. Also referred to herein as ‘reactions’.

36. Effects: Defined herein as the measurable properties of probe-target interactions or the consequences of the interactions (e.g., secondary reactions, ion channel opening or closing, etc.). These include, the following properties, for example:
i) the intensity of the nonlinear or fuindamental light.
ii) the wavelength or spectrum of the nonlinear or fundamental light.
iii) position of incidence of the fundamental light on the surface or substrate (e.g., for imaging).
iv) the time-course of either i), ii) or iii).
v) one or more combinations of i), ii), iii) and iv).

37. Time-course: Refers herein as the change in time of some measurable experimental such as light intensity or wavelength of light. Also referred to as ‘kinetics’ of some probe-target interaction, or probe-target-other component interaction for example.

38. Well-defined: In the context of ‘well-defined direction’, refers herein to the deterministic scattering of light (fundamental or nonlinear beams) from a substrate. By contrast, for example, fluorescence emission is emitted at somewhat random directions.

39. Sample: Contains the probes, targets or other molecules, particles or moieties under study by the invention. The sample contains at least one interface capable of generating the nonlinear optical light, with said interface comprised of at least one surface containing attached probes. Examples of components of samples include prisms, wells, microfluidics, substrates, buffer with targets, drugs in buffers, surfaces with attached probes. The terms ‘substrate’ and ‘surface’ are often used interchangeably herein. In some cases, the term ‘support’ can be construed to mean ‘surface’.

40. Modulator, Modulates: This term refers herein to any substance, moiety, molecule, biological component or compound which influences the kinetic or equilibrium properties of probe-target interactions (e.g., binding reaction). Modulators may change the rate of probe-target binding, the equilibrium constant of probe-target binding or, in general, enhance or reduce probe-target interactions. Examples of modulators are the following: inhibitors, drugs, small molecules, agonists and antagonists.

Indicators

The following section describes indicators in more detail. It was demonstrated in the art that an oxazole dye 4-[5-methoxyphenyl)-2-oxazoly-1]pyridinium methanesulfonate (also known as 4PyMPO-MeMs) is strongly second harmonic-active and chemically stable at neutral pH (Salafsky and Eisenthal, Chemical Physics Letters). Furthermore, the Stokes shift of the fluorescence which results from two-photon absorption is large so that the second harmonic beam can readily be separated from the fluorescence. Other dyes in this family have similar properties (J. H. Hall, 1992). EFISH (Electric-field induced second harmonic generation) can, for example, be used to determine if a candidate molecule or particle is nonlinearly active. Electric field induced second harmonic (EFISH) is well known in the field of nonlinear optics. This is a third order nonlinear optical effect, with the polarization source written as: P.sup.(2)(.omega..sub.3)=.chi..sup.(2)(-.omega..sub.3; .omega..sub.1, .omega..sub.2): E.sup..omega. 1 E.sup..omega.2. The effect can be used to measure the hyperpolarizabilty of molecules in solution by using a dc field to induce alignment in the medium, and allowing SHG to be observed. This is sometimes called the reorientational mechanism. These and other molecules, or assemblies of the molecules, can be used as indicators in the present invention:
5-(4-methoxphenyl)-2-(4-methoxyphenyl)-2-(4-pyridyl)oxazole
2-(4-methoxyphenyl)-5-(4-pyridyl)oxazole
2-(4-methoxyphenyl)-5-(4-pyridyl)oxadiazole
2-(4-methoxyphenyl)-5-(4-pyridyl)furan
2-(4-pyridyl)-4,5-dihydronapthol[1,2-d]-1,3-oxazole
5-Aryl-2-(4-pyridyl)-4-R-oxazole where R is a hydrogen atom, methyl group, ethyl group or other akyl group.
2-(4-pyridyl)cycloalkano[d]oxazole
2-(4-pyridyl)phenanthreno [9,10-d]-1,3-oxazole
6-Methoxy-4,4-dimethyl-2-(4-pyridyl)indeno[2,1-d]oxazole
4,5-Dihydro-7-methoxy-2-(4-pyridyl)napthol[1,2-d]-1,3-oxazole

Other molecules or molecules of the following families which can be used as indicators, include:
Merocyanines
Stilbenes
Indodicarbocyanines
Hemicyanines
Stilbazims
Azo dyes
Cyanines
Stryryl-based dyes
Methylene blue
Diaminobenzene compounds
Polyenes
Diazostilbenes
Tricyanovinyl aniline
Tricyanovinyl azo
Melamines
Phenothiazine-stilbazole
Polyimide
Sulphonyl-substituted azobenzenes
Indandione-1,3-pyidinium betaine
Fluorescein
Benzooxazole
Perylene
Polymethacrylates
Oxonol

Derivatized Particle Indicators

A solid microparticle or a nanoparticle of size nanometers to microns in scale including, but not limited to, a sphere (latex, polystyrene, silica, etc.) or a strip, offers a surface area which can be derivatized with a nonlinear-active moiety via chemical or electrostatic means so that the entire object has a much higher hyperpolarizability than can be obtained otherwise. For instance, nonlinear-active dyes can be ordered on silica bead surfaces via electrostatic interactions (dye is positively charged, silica surface is negatively charged) and the entire bead, if derivatized with target-reactive linkers, can then function as an indicator. If the nonlinear active moieties can be aligned on the solid surface so that phase interference between moieties is small, the overall hyperpolarizability will scale nonlinearly (eg., quadratically) in their number. The solid particle can vary in shape and its size can range from nanometers to microns in scale. Examples of the particles to be used include, but are not limited to, polystyrene beads and silica beads, both readily commercially available.

a. Covalent Attachment

The solid particles can be surface-derivatized using a variety of chemistries available in the art. Nonlinear-active moieties can be covalently coupled either to the solid particles or to a derivatized layer.

For instance, polystyrene beads can be derivatized with dextran, lactose or amines (the latter case for example, via chloromethyl groups with ethylenediamine). Silica can be derivatized using organofunctional silanes, for example using trichlorosilanes or other functional silanes (such as methoxy, amine, or other functional groups), to produce surfaces with a variety of chemical functionalities. The surfaces of the derivatized beads can then be reacted with a nonlinear active moiety via appropriate chemistry to produce the indicator.

b. Electrostatic Attachment

Nonlinear active moieties can also be electrostatically bound to a micron- or nanometer-sized particle surface to produce indicators with large hyperpolarizabilities. A charged nonlinear active moiety, an organic dye for example, can be oriented at a counter-charged microparticle surface, thus allowing for a net hyperpolarizability of the object when using an appropriate geometry. An example of an appropriate geometry is a microparticle sphere where the diameter is approximately the wavelength of the fundamental light, i.e. from tens of nanometers to microns so that destructive phase interference between nonlinear active moieties on opposing faces of the sphere is minimized. The hyperpolarizability of each dye at the spheres's surface, when integrated across the entire surface of the sphere of .about.wavelength of light size, is large and positive. For example, Silica beads (.about.200 nm, roughly spherical) are reacted with a low concentration of 3-aminopropyltrimethoxysilane or 3-aminooctyltrimethoxysilane so that only .about.5-10% of the surface silanols become covalently coupled to the silane agent. These amine groups are then reacted with the amine-reactive homobifunctional crosslinker Disuccinimidyl glutarate (DSG, Pierce Chemical) to create amine-reactive linkers on .about.5-10% of the bead surface. The beads are then incubated with 4-[5-methoxyphenyl)-2-oxazolyl]pyridinium methanesulfonate (also known as 4PyMPO-MeMs), a positively charged dye which binds electrostatically to the charged silanols on the surface and orients to some degree. The excess dye is removed from the beads by centrifugation. The electrostatic adsorption can be sufficiently high in some cases to immobilize the charged dye, even in the absence of a bulk concentration of it.

Derivatized Non-Centrosymmetric Nanocrystals, Nanoparticles, Clusters and Colloids

Art shows that metallic nanoparticles and clusters, ranging from about 1 nm to 25 or more microns in size, can be derivatized and conjugated to biomolecules for use in staining for electron microscopy, x-ray scattering and other applications. The art also shows that metal nanoparticles can exhibit extremely high hyperpolarizabilities (3, 5, 9). Another aspect of the present invention therefore is to use non-centrosymmetric metal nanocrystals or nanoparticles as indicators. A variety of shapes and sizes of metal or semiconductor nanoparticles are available in the art. A number of embodiments employing these metal particles are described herein.

Various combinations, in a wide variety of geometries, of linkers and metallic or semiconductor particles can be used to produce an indicator. The particles can be both centrosymmetric or non-centrosymmetric. If centrosymmetric, they must be joined together in clusters to create a composite particle which is overall non-centrosymmetric; or they must be greater than or equal to approximately 10% of the wavelength of the fundamental light used in the nonlinear optical technique.

In an alternate embodiment, metallic or semiconductor particles (either centrosymmetric or non-centrosymmetric) can be coupled to an SHG-active particle (such as oxazole, a stryrl dye, or some other molecule or particle). These resonantly enhancing particles are well known in the art to strongly increase the intensity of nonlinear light scattered from a nearby nonlinear active moiety. For example, gold nanoparticles have been used to strongly enhance the SH-activity of a styryl dye [14]. Because these resonantly enhancing particles are not themselves generating the nonlinear light, they can be centrosymmetric or non-centrosymmetric. They must be close enough to the SH-active moiety to create the resonant enhancement effect, which occurs through a dipole-dipole interaction; the distance between the two species for this effect to occur is typically on the order of angstroms to nanometers. The general resonance enhancement effect on nonlinear optical phenomena is discussed in the context of roughened silver surfaces in McAllister (1997) and Blanchard (1996). The resonantly enhancing particles are available commercially with a variety of surface chemistries amenable to coupling to an SH-active molecule such as oxazole (succinimidyl ester, maleimide, etc. offered by Molecules Probes, Eugene, Oreg.). Or the particle-nonlinear-active moiety complex can be constructed according to a number of schemes available in the art.

In an alternate embodiment, groups or chains of the metallic particles bound together via linking molecules can be used as indicators. For example, Au particles can be derivatized to chemically couple the particles together via art chemistry involving dimercapto-alkyl chains.

General Scheme of the Invention

A general scheme for measuring probe-target interactions or their effects using the present invention is as follows:

i) Illuminate the sample with light capable of undergoing second harmonic light; in the absence of either probes or targets, or both, the intensity and/or spectrum and/or timecourse of either or both intensity or spectrum of the second harmonic light can serve as a background or baseline.

ii) Add probes, targets, probes and targets, drugs, etc., or other components, which can modulate the probe-target interactions or their effects (at the same time or at separate times) and measure the resulting second harmonic light intensity and/or spectrum (or as a function of time, i.e., timecourse). This measured information serves as the signal for the desired interaction.

iii) A direct, optical read-out of the measured information can be performed or, optionally, the measured information can be modeled to determine, for example, kinetic or equilibrium properties of the probe-target interactions, with or without blocking agents, inhibitors, agonist, antagonist, etc.

### Preferred Embodiment

In a preferred embodiment of the invention, a microarray of PNA oligonucleotides is created on a glass or silica coverslip following the instructions and references therein of Hoffmann, R., et al. “Low scale multiple array synthesis and DNA hybridization of peptide nucleic acids” Pept. Proc. Am. Pept. Symp., 15th (1999) 233-234 or those found in Matysiak, S., Hauser, N. C., Wurtz, S. and Hoheisel, J. D. “Improved solid supports and spacer/linker systems for the synthesis of spatially addressable PNA-libraries” Nucleosides Nucleotides 18 (1999) 1289-1291. The buffer or solution in contact with the PNA oligonucleotides can be chosen from a range of those known in the art. The buffer or solution containing the target component (target oligonucleotides—not PNAs) will contain an indicator molecule. In the preferred embodiment, the indicator molecule will be 4-[5-methoxyphenyl)-2-oxazolyl]pyridinium methanesulfonate (also known as 4PyMPO-MeMs), dissolved in the solution containing the targets to be tested for hybridization at 1 mM concentration. (alternatively, the indicator can be added after the hybridization reaction has occurred). Hybridization solutions are found in the art. For example, the web site: cmgm.stanford.edu/pbrown/protocols gives detailed instructions for probe-target hybridization. These are the ‘probe’ oligonucleotides.

A mixture of target oligonucleotides is then added to a teflon chamber solution in contact with the coverslip, with the coverslip coupled to a Dove prism by an index-matching fluid or gel. If the target nucleotide is complementary to a particular probe (with a base-pair sequence at a specific and known location), a hybridization binding reaction occurs between the probe and target sequences at that location. Detailed procedures in the art exist for the hybridization reaction, for instance, the web site cmgm.stanford.edu/pbrown/protocols lists one example. The non-bound oligonucleotides are left in bulk solution. The hybridization results in a change in the surface charge and potential due to the negatively charged phosphate groups on the target oligos, which, in turn, leads to a change in the polarization of the indicator and the intensity of the second harmonic signal. This signal is quantitatively modeled using a Langmuir adsorption curve to determine the concentration of hybridized probe-target duplex oligonucleotides using software and a PC.

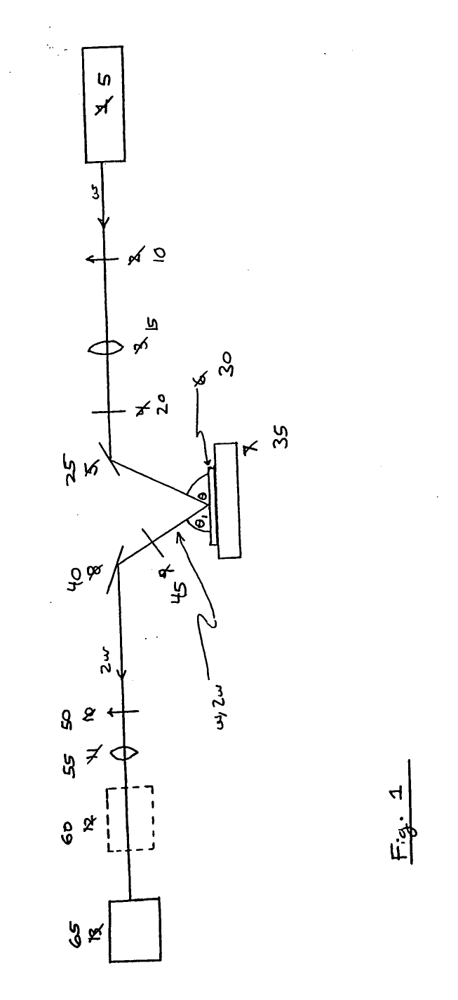

FIG. 1 illustrates the nonlinear optical apparatus. A femtosecond pulsed laser (5) (Spectra-Physics Corp.) operating at 800 nm at 80 MHz with sub-100 fs pulses at >0.5 W average power is used as the source of the fundamental light [alternatively, a 10W Argon ion laser (Coherent Corp.) can be used to pump a Ti:sapphire oscillator (Lexel Corp.) to produce the femtosecond or picosecond pulses of light]. The polarization of the fundamental can be optionally selected using a half-wave plate (10) (Melles Griot, 16 MLB 751) and focused by a lens (15) on to a color filter (20) (CVI Corp., LP 780) designed to pass the fundamental light but block the second harmonic radiation. The fundamental is then reflected from a mirror (25) and impinges at a specific location and with a specific angle on the sample surface (30). The beam diameter at the substrate surface is about 100 microns. The mirror (25) is scanned using a galvanometer-controlled mirror scanner, a rotating polygonal mirror scanner, a Bragg diffractor, acousto-optic deflector, or other means known in the art to allow control of a mirror's position. For instance, the incident angle and direction of the fundamental light on the sample surface can be varied in a known manner by the use of a precision adjustable mirror mount and a polygonal mirror using a Piezoelectric mirror mount 17 ASM 001—Melles-Griot and Mirror O₂ MLQ 011/003, Melles-Griot) and driving it through the use of a stepper driven motor (17PCSOO 1 and 17PCCOO 1 Melles-Griot) and PC control. A galvanometer mirror or any other PC-controllable beam deflection optic can be used. For example, 16-bit galvo positioners are available from General Scanning, Inc. and Cambridge Research which use closed loop servo-control systems to achieve precise alignment control.

The silica sample surface (30) is mounted on an x-y translation stage (35) (made from stacked linear stages, Newport Corp. PM500-L and computer controlled) to select a specific location on the surface for generation of the second harmonic beam. The sample surface (30) is enclosed and the surface in contact with liquid. The fundamental light is filtered using a color filter (Schott) leaving only the second harmonic light.

The second harmonic is reflected from mirror (40) (For example: 01 MFG 033/023, Melles-Griot Corp.), sent through a pass-filter (45) to pass the second harmonic while blocking the fundamental, and its polarization selected, if necessary, by a polarizing optic (50), then focusing the beam using a lens (11) onto a cooled CCD array detector (S7015 Hamamatsu) (13) where it impinges on a portion of the imaging sensors. The sensors produce a voltage signal proportional to the amount of impinging light; the monochromator can be used to select wavelengths or spectral bands for measurement. Since the beam diameter will typically be larger than a single element in the microarray, an XY-location for the center of the beam can be determined from the array of sensors in the CCD detector and standard image processing software. Spatial locations on the array surface can be correlated with specific CCD array sensor elements, and therefore a map of the intensity of nonlinear light vs. the microarray surface location can be determined. Real-time data can also be obtained in the same manner, either at a single region within the array, or back-and-forth scanning over time between regions so as to sample the same regions over a period of time. In this manner, an intensity image and its time-dependence can be acquired for any or all regions in the microarray. Given a fixed detector and incident angle and direction and stage position, the position of the reflecting mirror can be used to determine the region of the array under illumination since the nonlinear beam will have a well-defined direction with respect to the surface. Controlling the translation stage, a mirror scanner or both and correlating their positions with the measured signal of the elements (photodiodes) of a CCD array is disclosed in U.S. Pat. No. 6,084,991.

By translating either the stage or changing the incident position of the fundamental light, or some combination thereof, an image of second harmonic intensity from the entire array surface is built up, assigning intensity of second harmonic light to different regions or elements within the array using a standard software program such as Labview (Labview, Inc.) or some other custom-designed software. The intensity of the second harmonic light will be proportional to the amount of target on the surface and therefore, to the amount of hybridization between the target and the surface-attached probes. The negative charge of the phosphate groups on the target oligos which hybridize to the PNA probes will make the surface electric charge density more negative; this in turn, will increase the amount of polarized indicator near the interface, resulting in an increase in second harmonic radiation intensity. The relationship between the amount of hybridized target with measured second harmonic intensity is made quantitative with the known charge density of the surface with probes (and without targets) and the electric charge of the targets.

### Alternative Embodiments

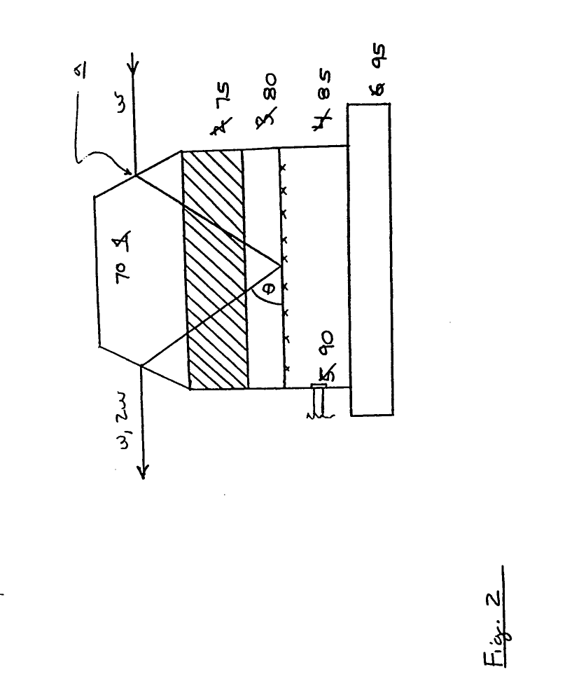

In an alternative embodiment, the microarray can be in contact with, attached to, or directly patterned on, a prism capable of allowing total internal reflection at the interface containing the probes. Thus, in this mode, the fundamental beam would undergo total internal reflection at the interface containing the probes and its evanescent wave would be used to generate the nonlinear light. FIG. 2 illustrates an embodiment of this type. In FIG. 2, an index matching material or liquid (75) is used to couple the prism (70) to a substrate containing the microarray (80) in contact with solution containing targets (85), whereby total internal reflection occurs at the interface between material (80) and solution (85). The prism material can be, for example, BK7 type glass (Melles Griot) and the index matching material obtained commercially from Corning Corp. or Nye Corp.

In an alternative embodiment, the experimental set-up is as described in Salafsky and Eisenthal, 2000 and references set forth therein. A femtosecond pulsed laser (Mail-Tai, Spectra-Physics) is used as the source of fundamental light at 800 nm operating at 80 MHz with <200 fs pulses at 1 W average power. The laser beam is focused with a concave lens (Oriel) (spot size .about. 1 mm.sup.2) on to the entrance aperture of a Dove prism (Melles Griot, BK-7) which is mounted in a teflon holder and in contact with solution (10 mM phosphate buffer, pH 7) or distilled water. The beam undergoes total internal reflection (evanescent wave generation) within the prism and the fundamental and second harmonic beams emerge roughly collinearly from the exit aperture. A color filter is used to block the fundamental light while passing the second harmonic to a monochromator (2 nm bandwidth slit). The monochromator is scanned from 380-500 nm to detect the second harmonic spectrum. If necessary, the fundamental light wavelength can be tuned as well. A single photon counting detector and photomultiplier tube are used to detect the output of the monochromator and a PC with software are used to record the data and control the monochromator wavelength. A background second harmonic signal is measured.

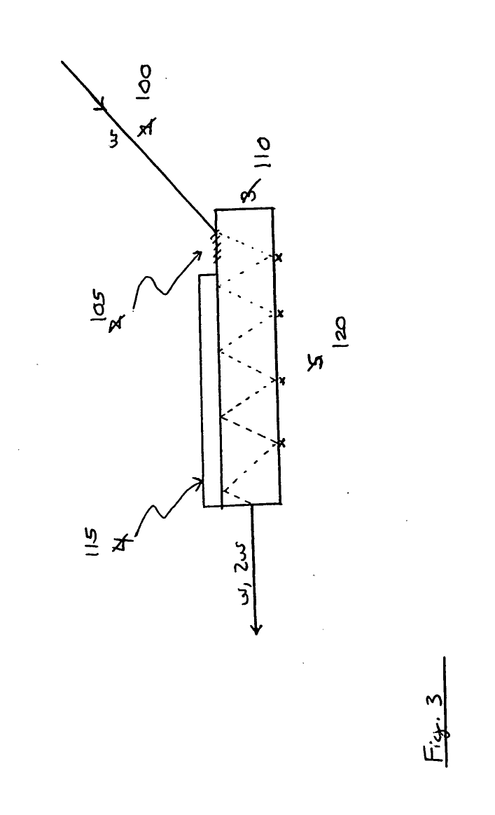

In an alternative embodiment, a planar waveguide structure 110 is used for the solid substrate (FIG. 3). In this embodiment, a thin layer of high index of refraction material 115 (the waveguide), such as TiO.sub.2 or Ta .sub.20.sub.5, is deposited on top of the substrate 110 (typically glass). A thin diffraction grating 115 is scribed into this waveguide and light from the laser 100 is coupled using this grating into the waveguide. Second harmonic light can be collected using lenses and filters and detected with either a PMT-type device or a CCD camera.

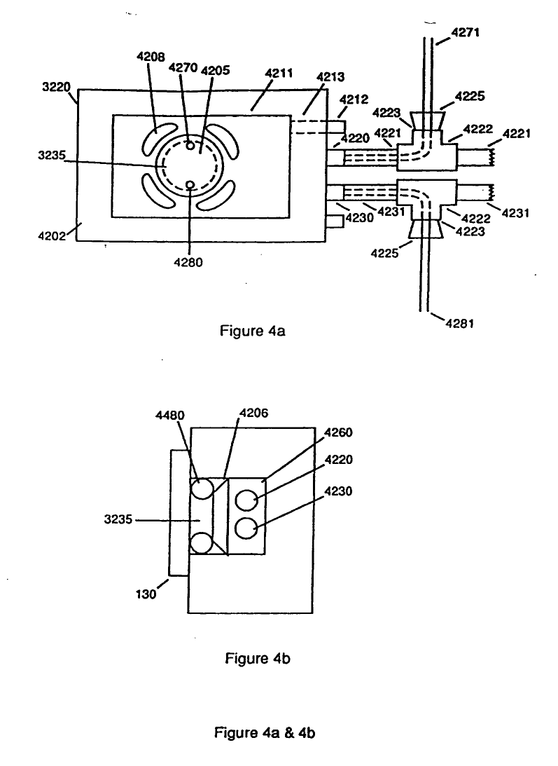

FIGS. 4a-4c illustrate an embodiment of a flow cell for carrying out probe-target reactions. The flow cell is 3220 is shown in detail. FIG. 4a is a front view, FIG. 4b is a cross sectional view, and FIG. 4c is a back view of the cavity. Referring to FIG. 4a, flow cell 3220 includes a cavity 3235 on a surface 4202 thereon. The depth of the cavity, for example, may be between about 10 and 1500.mu.m, but other depths may be used. Typically, the surface area of the cavity is greater than the size of the probe sample, which may be about 13.times. 13 mm. Inlet port 4220 and outlet port 4230 communicate with the cavity. In some embodiments, the ports may have a diameter of about 300 to 400.mu.m and are coupled to a refrigerated circulating bath via tubes 4221 and 4231, respectively, for controlling temperature in the cavity. The refrigerated bath circulates water at a specified temperature into and through the cavity.

A plurality of slots 4208 may be formed around the cavity to thermally isolate it from the rest of the flow cell body. Because the thermal mass of the flow cell is reduced, the temperature within the cavity is more efficiently and accurately controlled.

In some embodiments, a panel 4205 having a substantially flat surface divides the cavity into two subcavities. Panel 4205, for example, may be a light absorptive glass such as an RG1000 nm long pass filter. The high absorbance of the RG 1000 glass across the visible spectrum (surface emissivity of RG 1000 is not detectable at any wavelengths below 700 nm) substantially suppresses any background luminescence that may be excited by the incident wavelength. The polished flat surface of the light-absorbing glass also reduces scattering of incident light, lessening the burden of filtering stray light at the incident wavelength. The glass also provides a durable medium for subdividing the cavity since it is relatively immune to corrosion in the high salt environment common in DNA hybridization experiments or other chemical reactions.

Panel 4205 may be mounted to the flow cell by a plurality of screws, clips, RTV silicone cement, or other adhesives. Referring to FIG. 4b, subcavity 4260, which contains inlet port 4220 and outlet port 4230, is sealed by panel 4205. Accordingly, water from the refrigerated bath is isolated from cavity 3235. This design provides separate cavities for conducting chemical reaction and controlling temperature. Since the cavity for controlling temperature is directly below the reaction cavity, the temperature parameter of the reaction is controlled more effectively.

In an alternative embodiment, DNA or RNA are used instead of peptide nucleic acids.

In an alternative embodiment, a protein such as a GPCR protein is reconstituted into liposomes at near close-packed density and the vesicles are attached or adsorb to a surface such as glass, using procedures known to one skilled in the art. An indicator such as 4-[5-methoxyphenyl)-2-oxazolyl]pyridinium methanesulfonate (also known as 4PyMPO-MeMs) is dissolved in the buffer containing the liposomes. A background signal with the indicators and vesicles is measured at the surface using evanescent wave excitation with the fundamental beam; the second harmonic is wavelength-resolved using a monochromator and its intensity measured by a photomultiplier tube. A drug candidate is added to the solution; binding or a conformational change induced by binding of the drug candidate to the GPCR results in a change in the second harmonic signal, providing an optical read-out of the reaction. A control can be performed, if desired, to assign measured changes in nonlinear optical properties to binding or activation of components to a given receptor. For example, a component which is known to bind to a given receptor but not to produce a conformational change can be used as a control of the label reaction. A known agonist can be used to produce a calibration of the SHG intensity read-out in time and this can be used to compare against the read-out measured with unknown test candidates in drug screening.

In an alternative embodiment, the soluble extracellular fragment of an integrin protein such as αvβ3 is attached to a PEG-derivatized (PEG: polyeythylene glycol) glass surface via cysteines or amines. The αvβ₃ integrin fragment is produced according to procedures contained in the article by Takagi et al. [Cell, v. 110, p. 599-611, 2002, “Global Conformational Rearrangement in Integrin Extracellular Domains in Outside-In and Inside-Out Signaling”] and in references therein. The PEG materials such as H₂N-PEG-NH2 (MW ˜3400) can be purchased from Nektar, Inc. An indicator such as 4-[5-methoxyphenyl)-2-oxazolyl]pyridinium methanesulfonate (also known as 4PyMPO-MeMs) is dissolved in the buffer. SHG is measured using an evanescent wave reflection from the glass in contact with an index-matching fluid and clamped to a prism. The fundamental beam is passed through the prism in such a way that it forms an evanescent wave at the glass-buffer interface. Most of the background SHG will be due to the indicators. Upon binding or conformational change of a drug candidate to the integrin fragment, the density and/or orientation of the indicators will change at the interface with the integrin, providing a real-time readout of the interaction. Controls can be performed to eliminate artifacts.

Substrate 130 is mated to surface 4202 and seals cavity 3235. Preferably, the probe array on the substrate is contained in cavity 3235 when the substrate is mated to the flow cell. In some embodiments, an O-ring 4480 or other sealing material may be provided to improve mating between the substrate and flow cell. Optionally, edge 4206 of panel 4205 is beveled to allow for the use of a larger seal cross section to improve mating without increasing the volume of the cavity. In some instances, it is desirable to maintain the cavity volume as small as possible so as to control reaction parameters, such as temperature or concentration of chemicals more accurately. In additional, waste may be reduced since smaller volume requires smaller amount of material to perform the experiment.

Referring back to FIG. 4a, a groove 4211 is optionally formed on surface 4202. The groove, for example, may be about 2 mm deep and 2 mm wide. In one embodiment, groove 4211 is covered by the substrate when it is mounted on surface 4202. The groove communicates with channel 4213 and vacuum fitting 4212 which is connected to a vacuum pump. The vacuum pump creates a vacuum in the groove that causes the substrate to adhere to surface 4202. Optionally, one or more gaskets may be provided to improve the sealing between the flow cell and substrate.

FIG. 4d illustrates an alternative technique for mating the substrate to the flow cell. When mounted to the flow cell, a panel 4290 exerts a force that is sufficient to immobilize substrate 130 located therebetween. Panel 4290, for example, may be mounted by a plurality of screws 4291, clips, clamps, pins, or other mounting devices. In some embodiments, panel 4290 includes an opening 4295 for exposing the sample to the incident light. Opening 4295 may optionally be covered with a glass or other substantially transparent or translucent materials. Alternatively, panel 4290 may be composed of a substantially transparent or translucent material.

In reference to FIG. 4a, panel 4205 includes ports 4270 and 4280 that communicate with subcavity 3235. A tube 4271 is connected to port 4270 and a tube 4281 is connected to port 4280. Tubes 4271 and 4281 are inserted through tubes 4221 and 4231, respectively, by connectors 4222. Connectors 4222, for example, may be T-connectors, each having a seal 4225 located at opening 4223. Seal 4225 prevents the water from the refrigerated bath from leaking out through the connector. It will be understood that other configurations, such as providing additional ports similar to ports 4220 and 4230, may be employed.

Tubes 4271 and 4281 allow selected fluids to be introduced into or circulated through the cavity. In some embodiments, tubes 4271 and 4281 may be connected to a pump for circulating fluids through the cavity. In one embodiment, tubes 4271 and 4281 are connected to an agitation system that agitates and circulates fluids through the cavity.

Referring to FIG. 4c, a groove 4215 is optionally formed on the surface 4203 of the flow cell. The dimensions of groove, for example, may be about 2 mm deep and 2 mm wide. According to one embodiment, surface 4203 is mated to the translation stage. Groove 4211 is covered by the translation stage when the flow cell is mated thereto. Groove 4215 communicates with channel 4217 and vacuum fitting 4216 which is connected to a vacuum pump. The pump creates a vacuum in groove 4215 and causes the surface 4203 to adhere to the translation stage. Optionally, additional grooves may be formed to increase the mating force. Alternatively, the flow cell may be mounted on the translation stage by screws, clips, pins, various types of adhesives, or other fastening techniques.

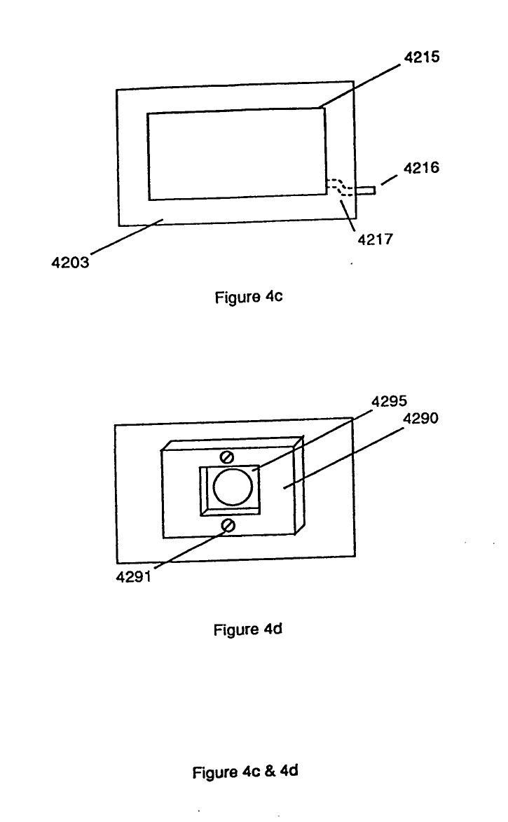

In a further alternative embodiment, a suspension of beads, cells, liposomes or other objects are the probes (130), or comprise probes attached thereto, as shown in FIG. 5. The scattered nonlinear light from such a sample—eg., an isotropic sample in which each individual beads or other objects are about a coherence length or farther apart—is generated in all directions with some distribution in intensity. Fundamental light is transmitted through the suspension (130) and the nonlinear radiation collected. A number of modes of collecting the scattered nonlinear light are available. For example, collection of the second harmonic can be in the forward direction (A), at a right angle to the fundamental light (B), or using an integrating sphere approach (C). Part C shows an integrating sphere 165 with the sample 150 placed inside. Fundamental light (145) enters the entrance port (170), passes through the sample (150), undergoes a reflection at the sphere wall, and is stopped by baffle (175). The scattered second harmonic light is collected from the sphere surface through exit port (155) and coupled out of the sphere by a fiber optic line (160). Beads can support phospholipid bilayers (eg., with membrane proteins) or probes such as proteins or nucleic acids can be attached to their surface. The beads provide a large amount of distributed surface area in the sample and can be a useful alternative to planar surface geometries, especially when the fundamental and nonlinear light is used in the transmission mode.

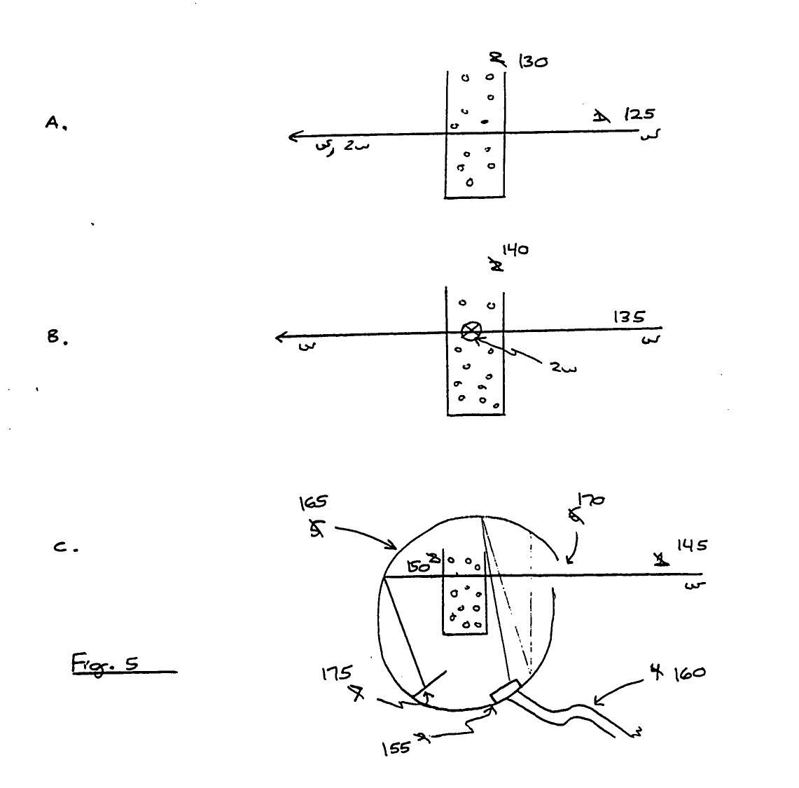

In an alternative embodiment (FIG. 6), the excitation light is tranformed from a point-like shape into some other shape using various optics. For instance, the point-like beam shape of the fundamental beam can be transformed into a line shape, useful for scanning the sample surface. However, because the intensity of the nonlinear beam depends on, among other factors, the intensity of the fundamental (typically a quadratic dependence on the fundamental intensity), this transformation will result in less nonlinear light intensity generated at a given location. To generate a line-shape in the fundamental (which can typically be a round point of 2 mm diameter), one can direct the fundamental beam into a microscope objective which has a magnification power of about 10 followed by a 150 mm achromat to collimate the beam as well known in the art and as disclosed in detail in U.S. Pat. No. 5,834,758. As shown in FIG. 6, the fundamental light 180 is a beam of typically 2-3 mm diameter. This beam is directed through a microscope objective 185. The objective, which has a magnification power of 10, expands the beam to about 30 mm. The beam then passes through a lens 190. The lens, which can be a 150 mm achromat, collimates the beam. Typically, the radial intensity of the expanded collimated beam has a Gaussian profile. To minimize intensity variations in the beam, a mask 195 can be inserted after lens 190 to mask the top and bottom of the beam, thereby passing only the central portion of the beam. In one embodiment, the mask passes a horizontal band that is about 7.5 mm. Thereafter, the beam passes through a cylindrical lens 200 having a horizontal cylinder axis, which can be a 100 mm f. 1. made by Melles Griot. The cylindrical lens expands the beam spot vertically. Alternatively, a hyperbolic lens can be used to expand the beam vertically while resulting in a flattened radial intensity distribution. From the cylindrical lens, the light passes through a lens 205. Optionally, a planar mirror can be inserted after the cylindrical lens to reflect the excitation light toward lens 205. To achieve a beam height of about 15 mm, the ratio of the focal lengths of the cylindrical lens 200 and lens 205 is approximately 1:2, thus magnifying the beam to about 15 mm. Lens 205, which in some embodiments is a 80 mm achromat, focuses the light to a line of about 15 mm.times.50 microns at the sample surface 210.

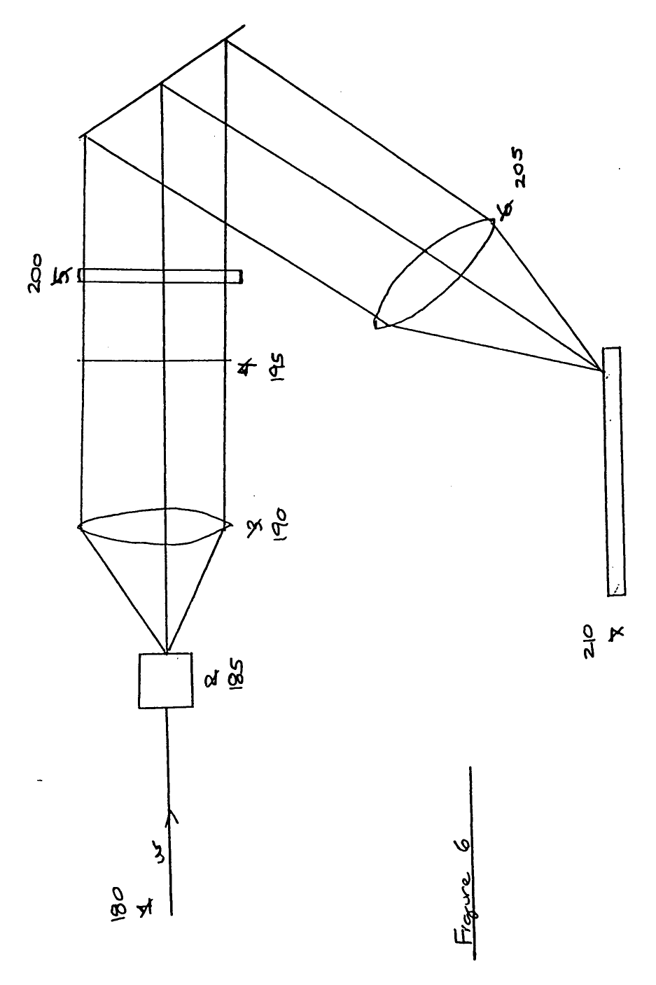

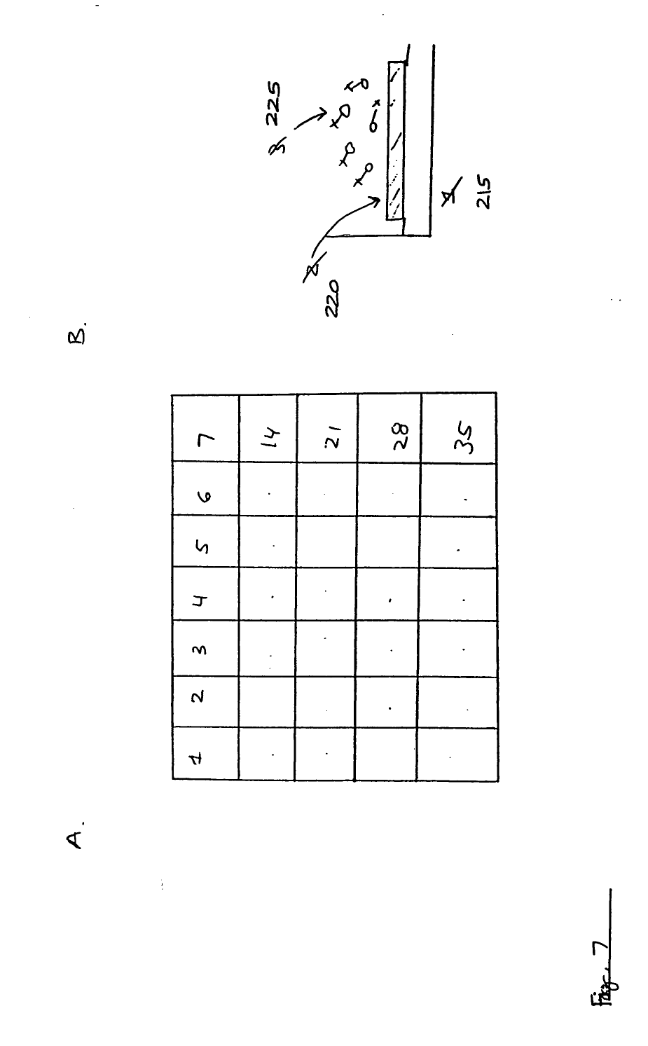

In an alternative embodiment shown in FIG. 7, probes patterned in a two-dimensional array (A, top view of array on surface) where each region on the surface—{1,35} in this example—can be a different oligonucleotide or protein sequence (or a combination of the same and different sequences) and labeled targets are used to detect binding. Part B shows a side-view of the sample surface (220) in a well (215) containing the targets (225) shown here as protein objects. The well can hold liquid or buffer and serves to physically separate the contents of the well from other parts of the substrate or other elements in a substrate array. The fundamental light can be multiplexed and each resultant beam can be guided by individual mirrors to simultaneously scan different lines or regions within the array, thus increasing even further the potential of the technique for high-throughput studies.

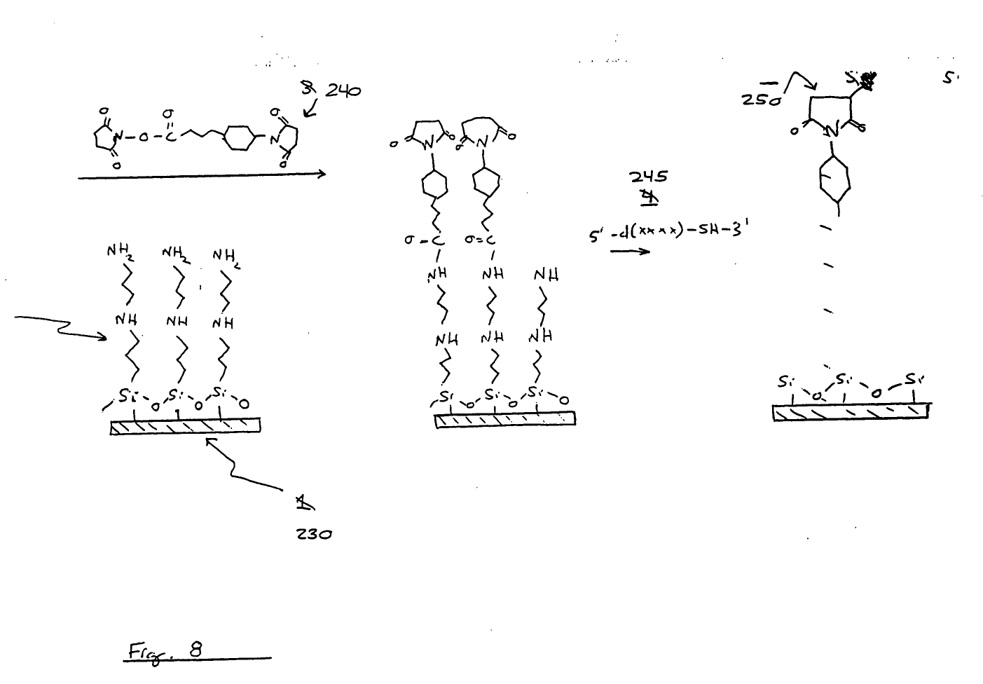

In an alternative embodiment, the method of Levicky et al. or the method of L. A. Chrisey et al. is used to attach the probe DNA to the substrate. In the method of Chrisey as illusrated in FIG. 8, a fused silica or oxidized silicon substrate is used (230) and derivatized with N-(2-aminoethyl)-3-aminopropyltrimethoxysilane (EDA) (235). In one embodiment, the EDA-modified surface is then treated with the heterobifunctional crosslinker (SMPB), whose succinimide ester moiety reacts with the primary amino group of EDA (240). A thiol-DNA oligomer subsequently (245) of base-pair sequence (xzzy) (where ‘xzzy’ represents the entire sequence) reacts with the maleimide portion of the SMPB crosslinker, to yield the covalently bound species shown (250).

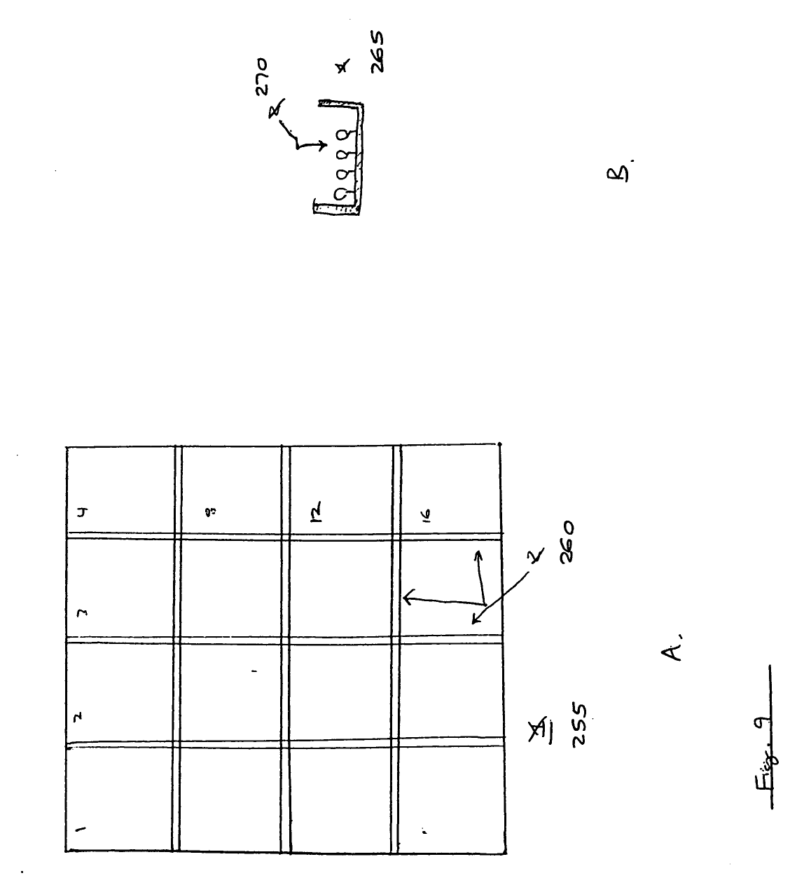

In an alternative embodiment, elements in the surface array are physically separated as illustrated in FIG. 9, allowing for different targets, target solutions, etc. to be added selectively to any or all of the elements. Part (A) is a top-view of the substrate (255) with partitions or walls (260) separating the different well regions—in this example, 16 wells. Part (B) shows a side-view of a well (265) with attached probes (270). Such arrays are commonly found in the art, such as the 96-well plates, etc. and are commercially available (Fisher Scientific, Inc. etc.)

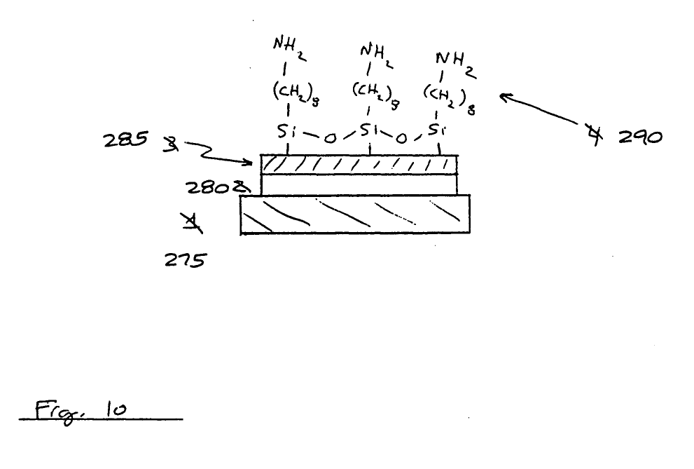

In an alternative embodiment, a glass substrate surface can be coated with a layer of a reflective metal such as silver. The metallic layer will increase nonlinear optical generation and collection. Biomolecules or other particles can be attached to derivatized layers built on top of the metal. For instance, the metal can be coated with a layer of silicon dioxide (SiO.sub.2), then with a layer of aminosilane such as 3-amino-octyl-trimethoxysilane. Oligonucleotides or polynucleotides can then be attached to the aminosilane layer using linkers which connect the 3′ or 5′ end of the oligo to the amine group. Alternatively, the oligos or polynucleotides can be adsorbed to the aminosilane layer. FIG. 10 illustrates an embodiment of this type where a glass substrate (275) is derivatized with a Ag layer (280). A thin coat of Sio.sub.2 is then deposited on top of the silver layer (285) and derivatized with the aminosilane (290).

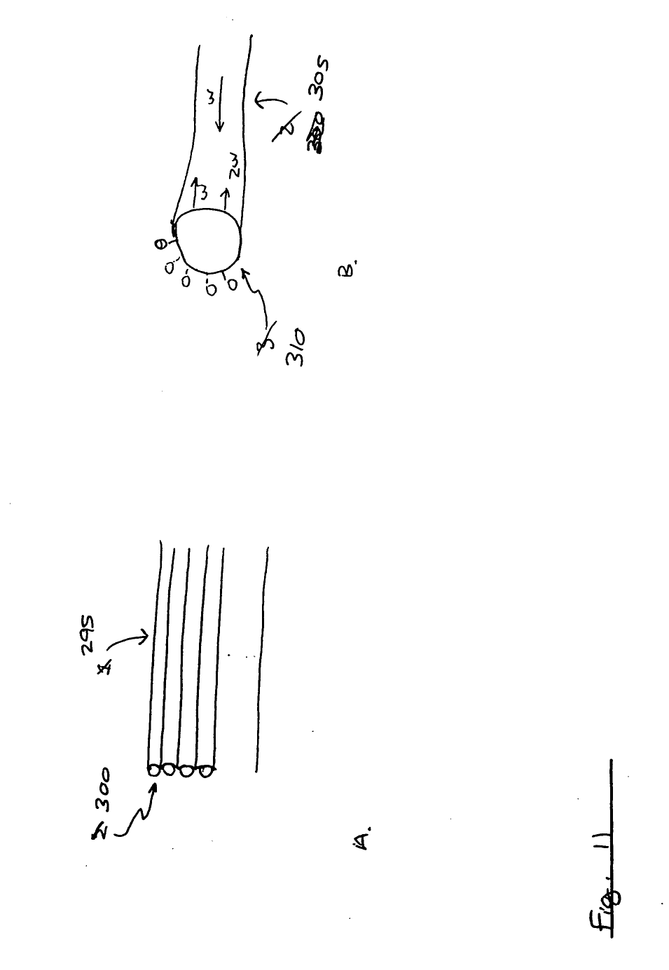

In an alternative embodiment, bead-based fiber-optic arrays can be used (ref. 34) in which light beams (eg., fundamental and second harmonic) travel via total internal reflection along the path of the fiber. The fundamental light is coupled into the bundle or individual optical fibers and second harmonic light is generated at the tip surface and collected back through the fiber. In this embodiment, individual optical fibers can be converted into DNA sensors by attaching a DNA probe to the distal tip (ref. 17,18) or by removing the cladding of the optical fiber and attaching the DNA probe to the outside of the core (ref. 19-22). Simple DNA arrays can be made from such optical fibers by physically bundling multiple fibers together (ref. 23). There are many variations on this theme, for example by selectively etching the distal-end cladding to create wells of different depths at the distal end of the fiber, where the tip of the fiber constitutes the bottom of the wells (ref. 24). Latex or silica beads can then be loaded into the wells (ref. 25). Fiber-optic oligonucleotide arrays can be prepared by attaching DNA probes to microspheres and then filling each well with a microsphere carrying a different DNA probe. Each different type of microsphere is tagged with a unique combination of fluorescent dyes or DNA probes either before or after probe attachment (refs. 26,27). ‘Zip codes’ for universal fabrication (ref. 29) and molecular beacons (refs. 28,30) for label-less detection can also be used with the optical sensor-beaded arrays. FIG. 11 illustrates a fiber-optic bundle array. Part (A) shows a bundle of fiber optic cables (295) with wells at the distals ends for placement of beads (300). Part (B) shows a close-up view of a single optical fiber. Fundamental light travels (o) toward the distal end with the bead (305). Some fundamental light is scattered back from the bead along with second harmonic light (2.omega and travels back through the fiber to the proximal end where an optical train and detection system (not shown) separates the fundamental radiation from the second harmonic radiation. Bead (310) is covered with attached probes.

In an alternative embodiment, the techniques of the present invention can be used to monitor the opening, closing or modulation of the behavior of ion channel receptors or other ionophores in cells, liposomes, supported bilayers, tissues in-vivo or in-vitro, or other membrane-based systems (for example, suspended in a medium as illustrated in FIG. 5 or attached to a solid substrate) via a change in the detected nonlinear optical radiation in response to a change in surface electric potential and charge density caused by ion flow through the ion channels. The ion channel receptors can be voltage-gated, chemically-gated or mechanically-gated. By following the opening and closing of the channels in response to agonists, antagonists, drugs, signalling molecules, a change in applied voltage across the sample, some combination of these, etc., the nonlinear optical technique can be used as a label-less means of determining the modes of molecular action on the mechanism of the ion channel proteins or receptors. The opening and closing of the ion-channels will—through the action of associated ions—change the electric potential at the membrane surface, and therefore polarization or orientation of water molecules, solvent or indicators near to it, thus allowing for the intensity of the nonlinear light to be used as a monitor of channel activity. The technique can also be used to time-resolve the opening or closing event and can be measured as a function of concentration of the agonist, antagonist, small-molecule, etc. In addition, a combination of compounds including, for example, agonists, antagonists, small molecules, etc. can be used in a single experiment to follow their combined effect on the ion channel receptors.

In this embodiment, the technique can be especially useful in monitoring the effects of drugs in neural cells and other cells (examples of other cells include, but are not limited to, muscle cells (smooth and striated), cardiac, endocrine, kidney cells, etc). Virtually all drugs that act on the nervous system (CNS or peripheral nervous system) produce their effects by modifying some step in the chemical synaptic transmission. Examples of compounds of interest include, but are not limited to: glycine, GABA (.gamma.-aminobutyric acid), glutamate, aspartate, other amino acids, acetylcholine, monoamines, dopamine, norepinephrine, 5-Hydroxytryptamine, peptides, nitric oxide, drugs that modify the action of these compounds, etc. The mode of action of a particular drug on the ion channel receptors is of interest in fundamental studies and for developing medicines, drugs or other therapeutic agents. For example, one can study the influence of benzodiazepines on GABA-benzodiazepine receptors, with or without the presence of other compounds such as barbituates, imidazopyridines, etc. Another example is the study of the many classes of antiarrythmic drugs which affect various channels in cardiac cells. The sample configuration can be set up in a variety of ways. For example, cells or liposomes under study can be in a suspension or solution and the fundamental can be passed through them (transmission mode) or they can be near (or attached to) an interface (e.g., a prism-solution interface) for evanescent mode interaction. Alternatively, tissues—for example in-vitro or as part of a living system (in-vivo) can be studied. Receptors can also be isolated and studied as part of a reconstituted system involving an artificial membrane. These examples are intended to be illustrative and not limiting of the scope of means in which this aspect of the present invention can be used. A background nonlinear optical signal can be measured. Addition of a target leads to a quantifiable change in the intensity of the nonlinear optical signal in time. The receptor that the target binds to can be part of the ion channel or some other receptor whose triggering results in a modulation of the ion channel's state or properties, for example ‘second messengers’ well known in the art. Addition of drugs, agonists or other components which can modulate the properites of the receptor-target interaction can be added to the sampel before addition or target, concomitantly with the target, during the target-receptor reaction or after the reaction has occurred. By comparing the nonlinear optical response of the receptor-target interaction with and without addition of the modulating components, one can determine whether these components have an influence on the receptor-target interaction, to what degree and of what type of influence (e.g., decreasing or increasing the target-receptor interaction, increasing or decreasing the rate at which the receptor-target interaction occurs, increasing or decreasing the rate of opening or closing of the ion channel, increasing or decreasing the permeability of the ion channel, etc.).

In an alternative embodiment, the detector (65) of the nonlinear radiation in FIG. 1 is a photomultiplier tube operated in single-photon counting mode. Photocurrent pulses can be voltage converted, amplified, subjected to discrimination using a Model SR445 Fast Preamplifier and Model SR 400 Discriminator (supplied by Stanford Research Systems, Inc.) and then sent to a counter (Model 3615 Hex Scaler supplied by Kinetic Systems). Photon counter gating and galvo control through a DAC output (Model 3112, 12-Bit DAC supplied by Kinetic Systems) can be synchronized using a digital delay/pulse generator (Model DG535 supplied by Stanford Research Systems, Inc.). Communication with a PC computer 29 can be accomplished using a parallel register (Model PR-604 supplied by DSP Technologies, Inc.), a CAMAC controller card (Model 6002, supplied by DSP Technologies, Inc.) and a PC adapter card (Model PC-004 supplied by DSP Technologies, Inc.).

According to another embodiment, detection of the nonlinear optical light is achieved using a charge coupled detector (CCD) in place of a photomultiplier tube or other photodetector. The CCD subsystem communicates with and is controlled by a data acquisition board installed in a computer. Data acquisition board may be of the type that is well known in the art such as a CIO-DAS 16/Jr manufactured by Computer Boards Inc. The data acquisition board and CCD subsystem, for example, may operate in the following manner. The data acquisition board controls the CCD integration period by sending a clock signal to the CCD subsystem. In one embodiment, the CCD subsystem sets the CCD integration period at 4096 clock periods. by changing the clock rate, the actual time in which the CCD integrates data can be manipulated.

During an integration period, each photodiode accumulates a charge proportional to the amount of light that reaches it. Upon termination of the integration period, the charges are transferred to the CCD's shift registers and a new integration period commences. The shift registers store the charges as voltages which represent the light pattern incident on the CCD array. The voltages are then transmitted at the clock rate to the data acquisition board, where they are digitized and stored in the computer's memory. In this manner, a strip of the sample is imaged during each integration period. Thereafter, a subsequent row is integrated until the sample is completely scanned.

In an alternative embodiment, an interference, notch-pass, bandpass, reflecting, or absorbant filter can be used in place of the filters in the Drawings in order to either pass or block the fundamental or nonlinear optical beams.

In an alternative embodiment, one is interested in finding drugs, antagonists, agonists or other species which block or reduce the binding of probes with targets—these compounds may be referred to as ‘inhibitors’. In this application, labeled targets are bound to probes at the interface. The inhibitors are added to the sample, and if the particular species being tested is successful in blocking or reducing the probe-target binding, the nonlinear optical light measured will change—the background radiation in this embodiment is due to target-probe binding; the displacement of the targets from the probes at the interface by the inhibitors leads to a change in the nonlinear optical light measured, for instance as a decrease in intensity of the nonlinear radiation generated by the interface or a wavelength shift in the nonlinear radiation spectrum.

In an alternative embodiment, the nonlinear spectrum of a sample is measured by measuring the nonlinear radiation (e.g., second harmonic radiation) at two or more spectral points or bands, using a monochromator, filter or other wavelength-selecting device to accomplish this.

In an alternative embodiment, a monochromator (60) can be placed before the detecting element in the device, in order to spectrally resolve the nonlinear optical radiation (FIG. 1).

In an alternative embodiment, a calibration of intensity of second harmonic light vs. concentration of probe-bound targets is obtained by using fluorescently tagged targets.

In an alternative embodiment, nucleic acid or PNA microarrays can be obtained commercially or constructed according to public literature (eg., http://cmgm.stanford.edu/pbrown/mguide/index.html). The surface chemistry to be used is that found in Chrisey, L. A. et al. (1996) in which oligonucleotides are attached to self-assembled monolayer silane films on fused silica slides. Silanization is accomplished via N-(2-aminoethyl)-3-aminopropyltrimethoxysilane.

In an alternative embodiment, oligonucleotides or PNAs can be attached to the solid substrate via light-directed synthesis (Fodor et al., 1997) or via chemical synthesis (e.g., Chrisey, L. A., 1996).

In an alternative embodiment, surfaces or microarrays microarrays of oligonucleotides or PNAs can be obtained commercially or constructed according to public literature (eg., http://cmgm.stanford.edu/pbrown/mgui- de/index.html).

DNA microarrays can be obtained commercially or constructed according to public literature (eg., http://cmgm.stanford.edu/pbrown/mgui- de/index.html). The surface chemistry to be used is that found in Chrisey, L. A. et al. (1996) in which oligonucleotides are attached to self-assembled monolayer silane films on fused silica slides. Silanization is done via N-(2-aminoethyl)-3-aminopropyltrimethoxysilane.a- nd Hoheisel, J. D. “Improved solid supports and spacer/linker systems for the synthesis of spatially addressable PNA-libraries” Nucleosides Nucleotides 18 (1999) 1289-1291 on glass or silica. The buffer or solution in contact with the PNA oligonucleotides can be chosen from a range of those known in the art. The solution containing the target component (target oligonucleotides—not PNAs) will contain an indicator molecule. In the preferred embodiment, the indicator molecule will be 4-[5-methoxyphenyl)-2-oxazolyl]pyridinium methanesulfonate (also known as 4PyMPO-MeMs), dissolved in the buffer solution at 1 mM concentration. Hybridization and wash solutions are found in the art. For example, the web site: cmgm.stanford.edu/pbrown/protocols gives detailed instructions for probe-target hybridization.

Microarrays can be mounted on an x-y translation stage and driven by personal computer (PC control) using a motorized translator (acquired from Oriel, Inc.) or using one of the many procedures in the art (eg., V. G. Cheung et al., 1999).

In an alternative embodiment, imaging techniques described in the art (Peleg et al. or Campagnola et al.) can be performed using indicators instead of the membrane-intercalating dyes used in the art. These imaging techniques iclecan be used to image solid surfaces, cell surfaces or other interface using indicators.

In an alternative embodiment, the indicators can be photoactivated or photomodulated with a beam of light (e.g., not the fundamental) such that, upon irradiation of the sample with the beam of light, the indicators become nonlinear optical active (or more or less nonlinear optical active). The beam of light can, for example, cleave a chemical bond (e.g., using UV light), well known in the art as ‘caged’ compounds.

In an alternative embodiment, the kinetics of some probe-target binding reaction are to be measured at some concentration of target. In this embodiment, the timecourse of the intensity and/or spectrum of the nonlinear optical light are measured. The measured information can be converted into a timecourse of bound target concentration (e.g., probe-target concentration in mM/s or .mu.M/s). Drugs or other enhancers or reducers, for example, of the probe-target binding equilibrium or kinetic rate of formation can be used so as to compare the effect of the added substance on the probe-target reactions.

In an alternative embodiment, the apparatus can be assembled into a user-closed product with a user-controlled interface (an LED panel, for example, or PC-based software) with the option of inserting and removing disposable substrates (e.g., biochips) with the attached probes.

In an alternative embodiment, the nonlinear optical measurements can be made in the presence of targets in solution, liquid or buffer in contact with the substrate with attached probes (e.g., no washing step is required to remove non-bound labelled targets).

In an alternative embodiment, channels (or microfluid) channels can be used to introduce the components into the sample cell via positive displacement, pumping, electrophoretic means or other means known in the art for manipulating the flow of components into and out of a reaction chamber.

In an alternative embodiment, a photodiode (65) in Drawing 1 is used as the light detection means.

In an alternative embodiment, the surface array can be in a fixed position and the incident light beam scanned across the surface using methods well known in the art, such as a galvanometer mirror or a polygonal mirror.

In an alternative embodiment, the scanning method can be a combination of both stage translation (x-y) and beam scanning, wherein the latter controls the incident position of the fundamental beam on the array surface.

In an alternative embodiment, a stop-flow mixing chamber is used to rapidly mix the components in the sample cell.

In an alternative embodiment, probe-target interactions with targets can be imaged on some surface such as a tissue surface, patterned cells on a surface, surface-attached probes (e.g., microarrays or arrays of DNA, protein, etc.) with the use of indicators, solvent molecules, water molecules, or the natural medium in-vivo which surrounds the surface; the imaging can occur in-vitro or in-vivo. In cases of in-vivo imaging, the imaging can be performed using endoscopes or other instruments known in the art for introducing and collect light in-vivo.

In an alternative embodiment, a biological probe-target binding reaction can be measured in the presence of agonists, antagonists, drugs, or small molecules which can modulate the binding strength (e.g., equilibrium constant) of the said probe-target binding reaction. This embodiment can be useful in many cases, for example when one would like to know the efficacy of a drug's ability to block a certain probe-target reaction for medical uses or basic research.

In an alternative embodiment, the nonlinear optical, surface-selective apparatus can comprise a unit without the light excitation source (e.g., with sample compartment, filters, detectors, monochromator, computer, interface, software, or other parts) so that the user can supply his own excitation source and adapt its use to the methods described herein.

In an alternative embodiment, the measurable information can be recorded in real time.

In an alternative embodiment, target-probe interactions can be measured in the presence of some modulator of the interactions—the modulator being, for example, a small molecule, drug, or other moiety, molecule or particle which changes in some way the target-probe interactions (e.g., blocks, inhibits, etc.). The modulator can be added before, during or after the time in which the probe-target interactions occur.

Various Configurations of an Apparatus Using the Surface-Selective Nonlinear Optical Technique for Detection of Probe-Target Reactions.

The apparatus for detection of the probe-target reactions or their effects can assume a variety of configurations. In its most simple form, the apparatus will comprise the following:
i) a source of the fundamental light
ii) a substrate or sample with surface-attached probes
iii) a detector for measuring the intensity of the second harmonic or other nonlinear optical beams.

More elaborate versions of the apparatus will employ, for example: a monochromator (for wavelength selection), a pass-filter, color filter, interference or other spectral filter (for wavelength selection or to separate the fundamental(s) from the higher harmonics), one or more polarizing optics, one or more mirrors or lenses for directing and focusing the beams, computer control, software, etc.

The mode of delivering or generating the nonlinear optical light (e.g., SHG) can be based on one or more of the following means: TIR (Total internal reflection), Fiber optics (with or without attached beads), Transmission (fundamental passes through the sample), Reflection (fundamental is reflected from the sample), scanning imaging (allows one to scan a sample), confocal imaging or scanning, resonance cavity for power build-up, multiple-pass set-up.

Measured information can take the form of a vector which can include one or more of the following parameters: {intensity of light (typically converted to a photovoltage by a PMT or photodiode), wavelength of light (determined with a monochromator and/or filters), time, substrate position (for array samples, for instance, where different sub-samples are encoded as function of substrate location and the fundamental is directed to various (x,y) locations}. Two general configurations of the apparatus are: image scanning (imaging of a substrate—intensity, wavelength, etc. as a function of x,y coordinate) and spectroscopic (measurement of the intensity, wavelength, etc. for some planar surface or for a suspension of cells, liposomes or other particles).

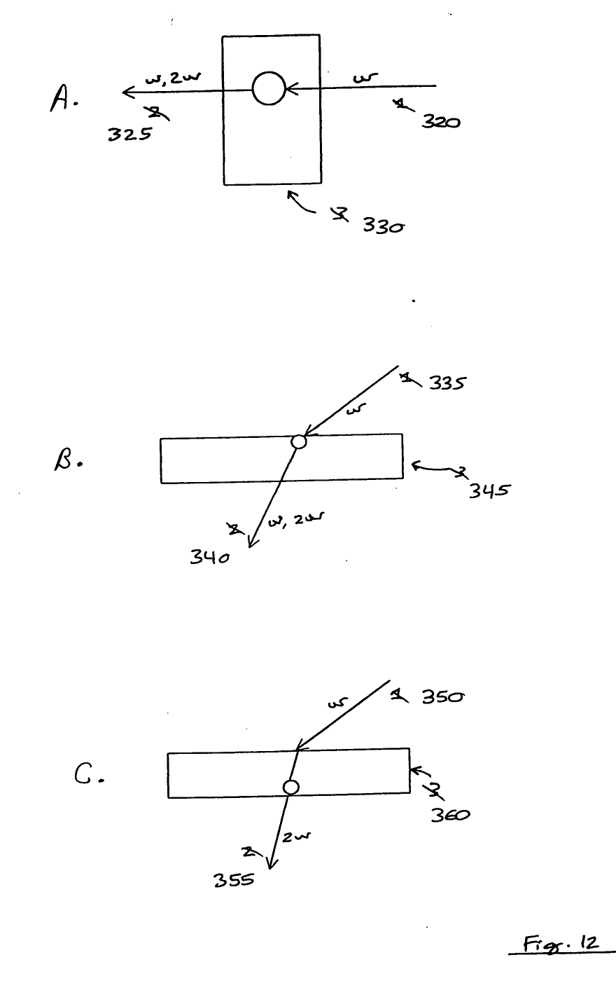

The fundamental beam can be delivered to the sample in a variety of ways. FIGS. 12-16 are schematics of various modes of delivering the fundamental and generating second harmonic beams. It is understood that in sum- or difference-frequency configurations, the fundamental beams will be comprised of two or more beams, and will generate, at the interfaces, the difference or sum frequency beams. For the purposes of illustration, only the second harmonic generation case is described in detail herein. Furthermore, it shall be understood that the sample cell 3 in all cases can be mounted on a translation stage (1-, 2-, or 3-dimensional degrees of freedom) for selecting precise locations of the interfacial interaction volume. The sample cell in all cases can be fitted with flow ports and tubes which can serve to introduce (or flush out) components such as molecules, particles, cells, etc.

Transmission

FIG. 12A is a schematic of a configuration relying on transmission of the fundamental and second harmonic beams. The fundamental 320 (.omega.) passes through the sample cell 330 and interacts within a volume element (denoted by the circle) in which are contained one or more interfaces capable of generating the second harmonic beam 325 (2.omega.). The fundamental and second harmonic beams are substantially co-linear as denoted by beam 325. The sample cell can contain suspended beads, particles, liposomes, biological cells, etc. in some medium, providing interfacial area capable of generating second harmonics in response to the fundamental beam. As shown, the second harmonic is detected co-linearly with the fundamental direction, but could alternatively be detected off-angle from the fundamental, for instance at 90.degree. to the fundamental beam.

FIG. 12B is a schematic of another configuration relying on transmission of the fundamental and second harmonic beams. The fundamental 335 is directed onto a sample cell 345 and the second harmonic waves are generated at the top surface—this surface can be derivatized with immobilized probes or with adsorbed particles, liposomes, cells, etc. The second harmonic waves 340 are generated within a volume element denoted by the circle at the interface between the top surface and the medium contained within cell.

FIG. 12C is a schematic of a configuration substantially similar to the one depicted in FIG. 2A except that the bottom surface of the sample cell 3, rather than the top, is used to generate the second harmonic waves.

Total Internal Reflection

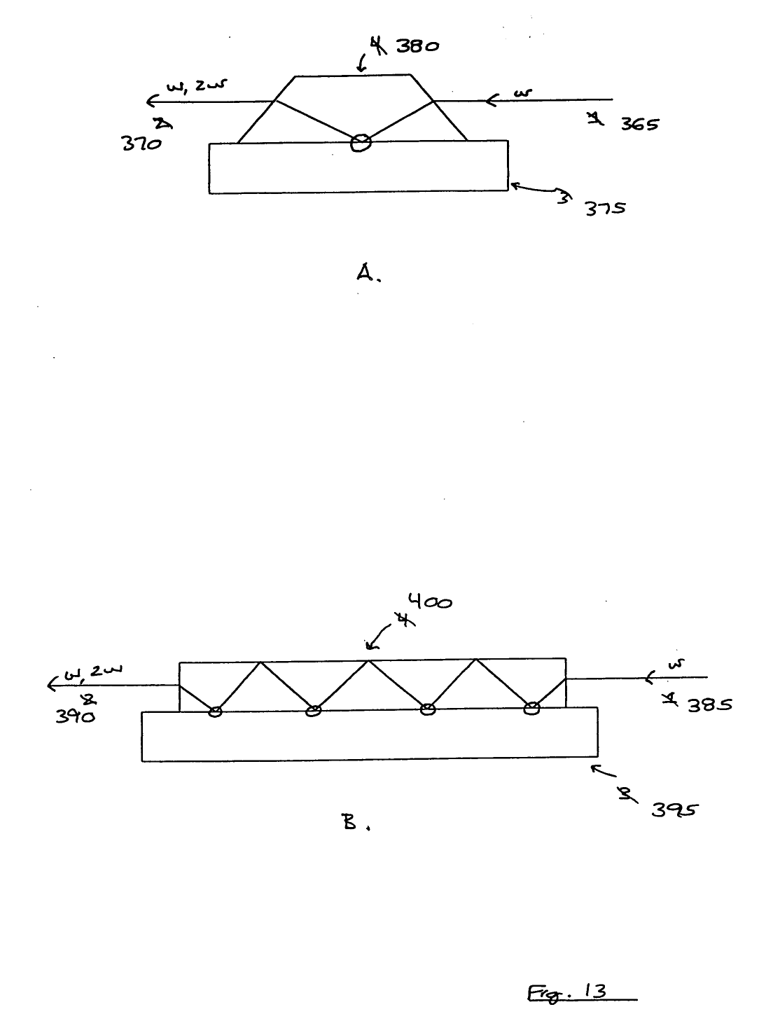

FIG. 13A is a schematic of a waveguide 4 capable of acting as a total internal reflection waveguide which refracts the fundamental 365 and directs it to a location at the interface between the waveguide 380 and a sample cell 375. At this location, denoted by the circle, the fundamental will generate the second harmonic waves and undergo total internal reflection; the second harmonic beam will propagate substantially colinearly with the fundamental and exit the prism 380. Waveguide 380 will typically be in contact with air. In this illustration, the waveguide 380 is a Dove prism.

FIG. 13B is a schematic of a configuration similar to the one depicted in FIG. 13A except that the waveguide 400 allows for multiple points of total internal reflection between the waveguide 4 and the sample cell 395, increasing the amount of second harmonic light generated from the fundamental beam.

Fiber Optic

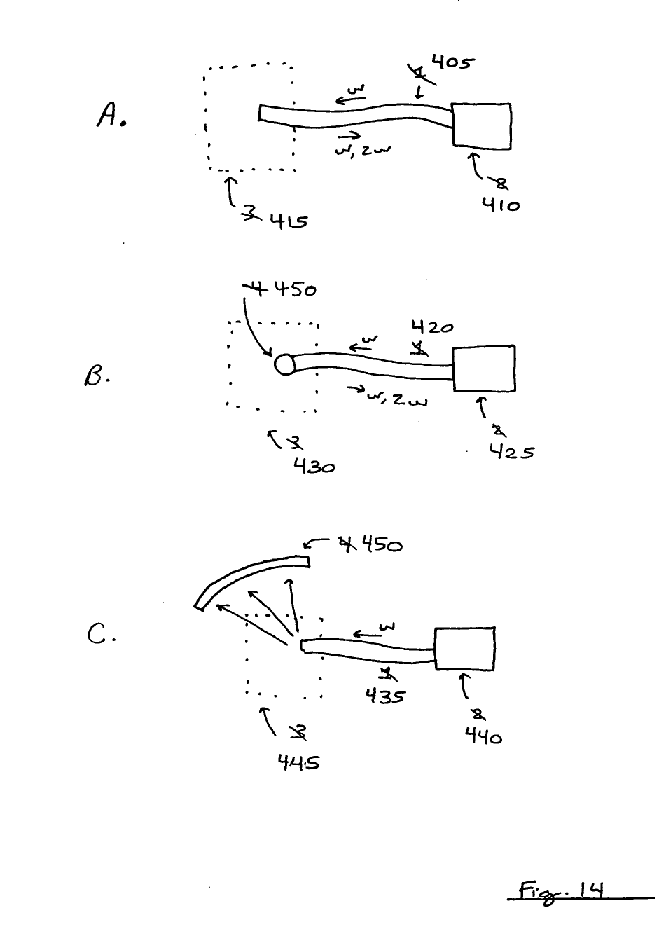

FIG. 14 depicts various configurations of a fiber optic means of delivering or collecting the fundamental or second harmonic beams. In FIG. 14A, the coupling element 410 between a source of the fundamental wave and the fiber optic is depicted. The fundamental, thus coupled into the fiber optic waveguide 405, proceeds to a sample cell 415. In FIG. 14A, the tip of the fiber can serve as the interface of interest capable of generating second harmonic waves, or the tip can serve merely to introduce the fundamental beam to the sample cell containing suspended cells, particles, etc. In FIG. 14A, the second harmonic light is collected back through the fiber optic.

FIG. 14B is identical to FIG. 14A except that a bead is attached to the tip of the fiber optic (according to means well known in the art). The bead can serve to both improve collection efficiency of the second harmonic light or be derivatized with probes or adsorbed species and presenting an interface with the medium of sample cell 425 capable of generating the second harmonic light.

FIG. 14C is identical to both FIGS. 14A and 14B except that collection of the second harmonic light is effected using a solid-angle detector 450.

Optical Resonance Cavity

An optical resonance cavity is defined between at least two reflective elements and has an intracavity light beam along an intracavity beam path. The optical cavity or resonator consists of two or more mirrored surfaces arranged so that the incident light can be trapped bouncing back and forth between the mirrors. In this way, the light inside the cavity can be many orders of magnitude more intense than the incident light. This phenomenon is well known and has been exploited in various ways (see, for example, Yariv A. “Introduction to Optical Electronics”, 2.sup.nd Ed., Holt, Reinhart and Winston, N.Y. 1976, Chapter 8). The sample cell can be present in the optical cavity or it can be outside the optical resonance cavity.

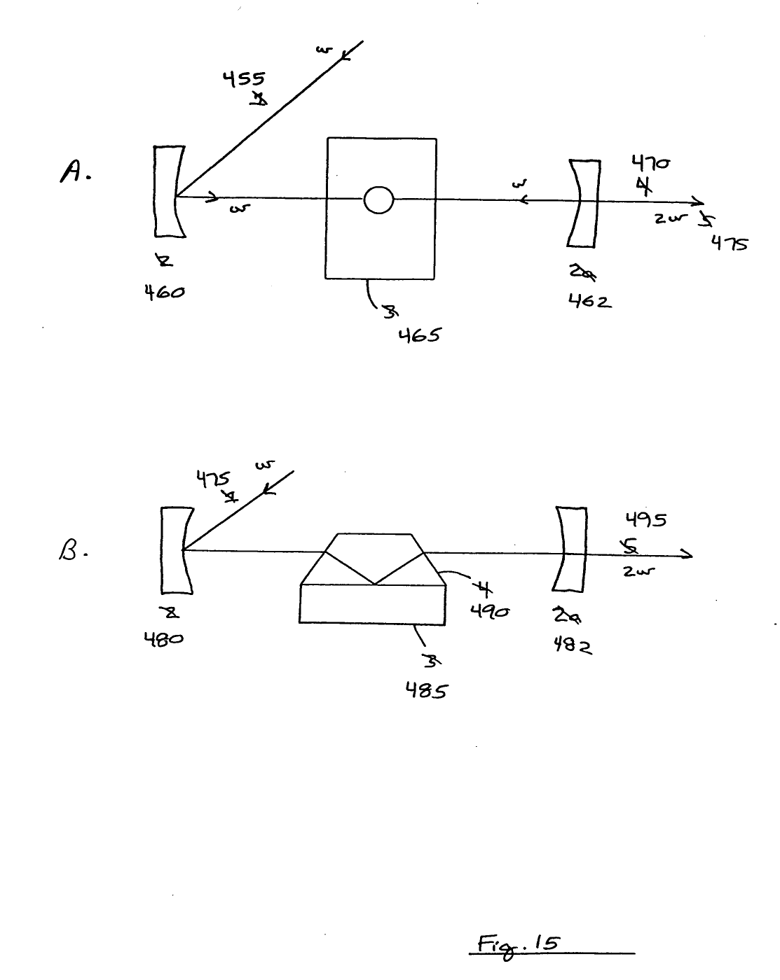

FIG. 15 is a schematic of an optical resonance power build-up cavity configuration. FIG. 15A is a schematic of an optical resonance cavity in which the sample cell 465 is positioned intracavity and the fundamental and second harmonic beams are transmitted through it—a useful configuration for sample cells containing suspended particles, cells, beads, etc. The fundamental beam 455 enters the optical resonance cavity at reflective optic 460 and builds up in power between reflective elements 460 and 462 (intracavity beam). Mirror 460 is preferably tilted (not perpendicular to the direction of the incident fundamental 455) to prevent direct reflection of the intracavity beam back into the light source. The natural reflectivity and transmisivity of 460 and 462 can be adjusted so that the fundamental builds up to a convenient level of power within the cavity. The fundamental generates second harmonic light in a volume element within the sample cell denoted by the circle. Reflective optic 460 can reflect the fundamental and the second harmonic, while reflective optic 462 will substantially reflect the fundamental but allow the pass-through of the second harmonic beam 475 which is subsequently detected. U.S. Pat. No. 5,432,610 (King et al.) describes a diode-pumped power build-up cavity for chemical sensing and it and the references it makes are hereby incorporated by reference herein.

FIG. 15B is a schematic of an optical resonance power build-up cavity configuration in which the fundamental beam 475 enters the optical cavity by reflection from optic 480. A second reflective optic element 482 defines the optical resonance cavity. Element 490 is a waveguide (such as a prism) in contact with the sample cell 485 and allows total internal reflection of the fundamental beam at the interface between the waveguide and sample cell surfaces, generating the second harmonic light. Element 482 substantially reflects the fundamental beam but passes through the second harmonic beam 495 which is subsequently detected.

Reflection

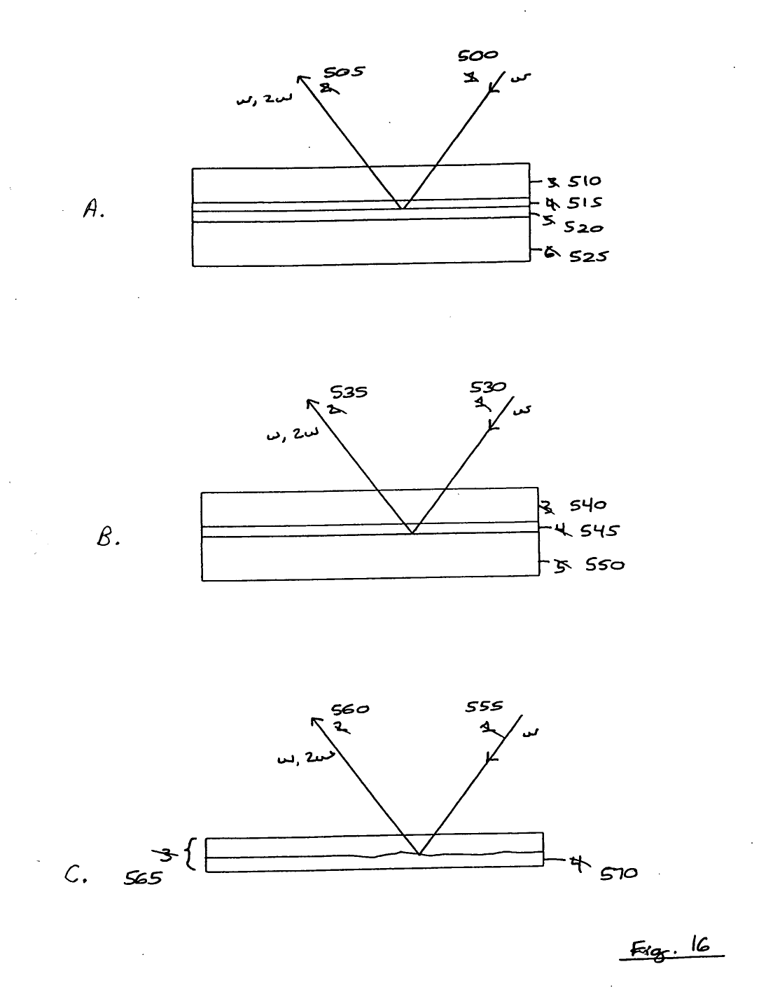

FIG. 16A is a schematic of a configuration involving reflection of the fundamental and second harmonic beams. A substrate 525 is coated with a thin layer of a reflective material 520, such as a metal, and on top of this is deposited at layer 515 suitable for attachment of the probes or adsorption of particles, cells, etc. (e.g., SiO.sub.2). This layer is in contact with the sample cell 510. The fundamental 500 passes through the sample cell 510 and generates a second harmonic wave at the interface between layers 515 and 520. The fundamental and second harmonic waves 505 are reflected back from the surface of layer 520.

FIG. 16B is substantially similar to FIG. 15A except that the second harmonic and fundamental beams are reflected 535 from the interface between the medium contained in sample cell 540 and layer 545. Layer 545 is reflective or partly reflective layer deposited on substrate 550 and is suitable for adsorption of particles, cells, etc. or attachment of probes.

FIG. 16C is a schematic illustrating that only the sample cell 565 need be used for a reflective geometry. The sample cell 565 is partly filled with some medium 570 and the fundamental and second harmonic beams are reflected 560 from the gas-liquid or vapor-liquid interface at the surface of 570.

Modes of Detection

Charge-coupled detectors (CCD) array detectors can be particularly useful when information is desired as a function of substrate location (x,y). CCDs comprise an array of pixels (i.e., photodiodes), each pixel of which can independently measuring light impinging on it. For a given apparatus geometry, nonlinear light arising from a particular substrate location (x,y) can be determined by measuring the intensity of nonlinear light impinging on a CCD array location (Q,R) some distance from the substrate—this can be determined because of the coherent, collimated (and generally co-propagating with the fundamental) nonlinear optical beam) compared with the spontaneous, stochastic and multidirectional nature of fluorescence emission. With a CCD array, one or more array elements {Q,R} in the detector will map to specific regions of a substrate surface, allowing for easy determination of information as a function of substrate location (x,y). Photodiode detector and photomultiplier tubes (PMTs), avalanche photodiodes, phototransistors, vacuum photodiodes or other detectors known in the art for converting incident light to an electrical signal (i.e., current, voltage, etc.) can also be used to detect light intensities. For CCD detector, the CCD communicates with and is controlled by a data acquisition board installed in the apparatus computer. The data acquisition board can be of the type that is well known in the art such as a CIO-DAS 16/Jr manufactured by Computer Boards Inc. The data acquisition board and CCD subsystem, for example, can operate in the following manner. The data acquisition board controls the CCD integration period by sending a clock signal to the CCD subsystem. In one embodiment, the CCD subsystem sets the CCD intregration period at 4096 clock periods. By changing the clock rate, the actual time in which the CCD integrates data can be manipulated. During an integration period, each photodiode accumulates a charge proportional to the amount of light that reaches it. Upon termination of the integration period, the charge is transferred to the CCD's shift registers and a new integration period commences. The shift registers store the charges as voltages which represent the light pattern incident on the CCD array. The voltages are then trasmitted at the clock rate to the data acquisition board, where they are digitized and stored in the computer's memory. In this manner, a strip of the sample is imaged during each integration period. Thereafter, a subsequent row is integrated until the sample is completely scanned.

Sample Substrates and Sample Cells

Sample substrates and cells can take a variety of forms drawing from, but not limited to, one or more of the following characteristics: fully sealed, sealed or unsealed and connected to flow cells and pumps, integrated substrates with a total internal reflection prism allowing for evanescent generation of the nonlinear beam, integrated substrates with a resonant cavity for fundamental power build-up, an optical set-up allowing for multiple passes of the fundamental for increased nonlinear response, sample cells containing suspended biological cells, particles, beads, etc.

Data Analysis

Data analysis operates on the vectors of information measured by the detector. The information can be time-dependent and kinetic. It can be dependent on the concentration of one or more biological components, which can be changed during a measurement or between measurements. It can also be dependent on wavelength, etc. In general, the intensity of nonlinear light will be transformed into a concentration or amount of a particular state (for example, the surface-associated concentration of a component or the amount of opened or closed ion-channels in cell membranes). In one example, the production of second harmonic light follows the equation:
(I.sub.SH).sup.0.5.varies.E.sub.2.omega.=A.chi..sup.(2)+B.PHI..sub.0.chi.- sup.(3) (1)
where I.sub.SH is the intensity of the second harmonic light, E.sub.2.omega.. is the electric-field amplitude of the second harmonic light, A and B are constants specific to a given interface and sample geometry, PHI..sub.0 is the electric surface potential, and chi..sup.(2) and .chi..sup.(3) are the second and third-order nonlinear susceptibility tensors. Surface binding reactions can follow a Langmuir-type equation:
   dN/dt=k.sub. 1 (C−N)/55.5*(N.sub.max-N)-k.sub.1N (2)
with N the amount of the targets binding to the surface (e.g., targets binding to probes), N.sub.max the maximum number of the binding species at the surface at equilibrium, k.sub. 1 the association rate constant, k.sub. 1 the dissociation rate constant, dN/dt the instantaneous rate of change of the amount of surface-bound targets and C the bulk concentration of the species. Modified Langmuir equations or other equations used in determining the amount of surface-adsorbed or surface-bound species in the art can also be used in the data analysis.

Another relevant equation is the Gouy-Chapman equation which relates the surface charge density to the surface electric potential and the bulk electrolyte concentration:
.sigma.=11.7(C).sup.0.5 sin h(19.5z.PHI.sub.0) (3)
with sigma. the surface charge density, C the bulk electrolyte concentration, z the charge of the electrolyte species and PHI..sub.0 the surface electric potential. Other equations of this type can also be used, such as the Gouy-Chapman-Stern equation which takes into account the finite size of the ions in the equation relating .PHI..sub.0 to sigma.

The details of the data analysis will depend on each specific case. If the polarization response due to a net charge on the surface—.chi..sup.(3)—is present, it can be subtracted out in making the measurement. Thus, the number of surface-bound species N can be directly calculated from the second harmonic intensity in this manner. Kinetics or equilibrium properties can be determined from N (at equilibrium or in real time) according, for example, to equation 2 and procedures well known in the art. There are a number of relevant papers in the art which describe this process in detail including, for example: J. S. Salafsky, K. B. Eisenthal, “Second Harmonic Spectroscopy: Detection and Orientation of Molecules at a Biomembrane Interface”, Chemical Physics Letters 2000, 319, 435 and Eisenthal, K. B. “Photochemistry and Photophysics of Liquid Interfaces by Second Harmonic Spectroscopy ” J. Phys. Chem. 1996, 100, 12997.

For probe-target processes which result directly or indirectly in changes in surface charge density (an example of the indirect type is in ion-channel experiments where a target binds to a probe, leading to the modulation of an ion channel's dynamics which leads, in turn, to the surface charge density, leading in turn to a change in polarization of water molecules, solvent molecules or indicators). In this case, the amount of charge at the surface for a given amount of N is determined by the net charge of the molecule and so N can be related to sigma. (surface charge density). Using a formula such as Equation 2, the surface electric potential can be determined and thereby used in Equation 1 to determine, in turn, the electric field amplitude or intensity of the second harmonic light. Thus, one can relate the the measured second harmonic intensity to the amount of surface-bound species (target). An example of this kind of data analysis is given in: J. S. Salafsy, K. B. Eisenthal, “Protein Adsorption at Interfaces Detected by Second Harmonic Generation”, Journal of Physical Chemistry B, 2000, 104(32), 7752-7755. Therefore, by using the measured data of the nonlinear optical radiation, one can determine N, the free energy of binding and the kinetics of the binding process according to procedures well known in the art.

### REFERENCES

U.S. Pat. No. 5,324,591 June, 1994 Georger, Jr. et al. U.S. Pat. No. 5,215,899 June, 1993 Dattagupta U.S. Pat. No. 6,030,787 February, 2000 Livak et al. U.S. Pat. No. 5,834,758 November, 1998 Trulson et al. U.S. Pat. No. 6,025,601 February, 2000 Trulson et al. U.S. Pat. No. 5,631,734 May, 1997 Stern et al. U.S. Pat. No. 6,084,991 July, 2000 Sampas U.S. Pat. No. 5,633,724 May, 1997 King et al. U.S. Pat. No. 6,121,983 September, 2000 Fork et al. U.S. Pat. No. 5,485,277 January, 1996 Foster et al. U.S. Pat. No. 5,324,633 June, 1994 Fodor et al. U.S. Pat. No. 6,124,102 September, 2000 Fodor et al. U.S. Pat. No. 5,847,400 December, 1998 Kain et al. U.S. Pat. No. 5,432,610 July, 1995 King et al. U.S. Pat. No. 5,320,814 June, 1994 Walt et al. U.S. Pat. No. 5,250,264 October, 1993 Walt et al. U.S. Pat. No. 5,298,741 March, 1994 Walt et al. U.S. Pat. No. 5,252,494 October, 1993 Walt et al. U.S. Pat. No. 6,023,540 February, 2000 Walt et al. U.S. Pat. No. 5,814,524 September, 1998 Walt et al. U.S. Pat. No. 5,244,813 September, 1993 Walt et al. U.S. Pat. No. 5,512,490 April, 1996 Walt et al. U.S. Pat. No. 6,095,555 July, 2000 Fiekowsky et al. U.S. Pat. No. 6,134,002 October, 2000 Stimson et al. U.S. Pat. No. 6,110,426 December, 1997 Shalon et al. U.S. Pat. No. 6,040,586 March, 2000 Slettnes et al.
1. J. S. Salafsky, K. B. Eisenthal, “Second Harmonic Spectroscopy: Detection and Orientation of Molecules at a Biomembrane Interface”, Chemical Physics Letters 2000, 319, 435-439.
2. J. S. Salafsy, K. B. Eisenthal, “Protein Adsorption at Interfaces Detected by Second Harmonic Generation”, Journal of Physical Chemistry B, 2000, 104, 7752-7755.
3. R. Ekins, F. W. Chu, Trends in Biotechnology, 1999, 17, 217.
4. G. MacBeath and S. L. Schreiber, “Printing Proteins as Microarrays for High-Throughput Function Determination”, Science 2000, 289, 1760-1763.
5. N.J. Harrick, “Internal Reflection Spectroscopy”, John Wiley & Sons, Inc., New York, 1979.
6. Groves, J. T., Ulman, N., Boxer, S. G., “Micropafterning fluid bilayers on solid supports”, Science, 1997, 275, 651-3.
7. Fodor et al., “Light-directed Spatially-addressable Parallel Chemical Synthesis,” Science, 1991, 251, 767-773.
8. Ramsay, G. DNA chips—states-of-the-art. Nature Biotechnology 1998, 16(1), 40-44.
9. Marshall, A.; Hodgson, J. DNA chips—an array of possibilities. Nature Biotechnology 1998, 16(1), 27-31.
10. S. C. Case-Green et al., Curr. Opin. Chem. Biol. 2 (1998), 404.
11. S. A. Fodor, Science 277 (1997), 393.
12. D. H. Duggan et al., Nature Genet. 21 (Suppl.) (1999), 10.
13. M. Schena et al., Science 270 (1995), 467.
41. P. O. Brown and D. Botstein, Nature Genet. 21 (Suppl.) (1999), 33.
42. L. McAllister et al., Am. J. Hum. Genet. 61 (Suppl.) (1997), 1387.
16. A. P. Blanchard et al., Biosens. And Bioelectron. 11 (1996) 687.
17. G. J. Zhang et al., Anal. Lett. 32, 2725 (1999).
18. F. Kleinjung et al., Anal. Chim. Acta 350, 51 (1997).
19. A. H. Uddin et al, Nucleic Acids Res. 25, 4139 (1997).
20. P. A. E. Piunno et al., Anal. Chem. 67, 2635 (1995).
21. F. F. Bier et al., Sens. Actuators B Chem. 38, 78 (1997).
22. A. P. Abel et al., Anal. Chem. 68, 2905 (1996).
23. J. A. Ferguson et al., Nature Biotechnol. 14, 1681 (1996).
24. P. Pantano and D. R. Walt, Chem. Mater. 8, 2832 (1996).
25. K. L. Michael et al., Anal. Chem. 70, 1242 (1998).
26. R. J. Fulton et al., Clin. Chem. 443, 1749 (1997).
27. M. Chee and D. R. Walt, unpublished results.
28. F. J. Steemers et al., Nature Biotechnol. 18, 91 (2000).
29. N. P. Gerry et al., J. Mol. Biol. 292, 251 (1999).
30. J. Liu and W. H. Tan, Anal. Chem. 71, 5054 (1999).
31. D. R. Walt, Science 287, (2000) 451.
[14] “Nonlinear optical measurement of membrane potential around single molecules at selected cellular sites”, G. Peleg, A. Lewis, M. Linial, L. M. Loew, Proc. Natl. Acad. Sci. V. 96, 1999, 6700-6704.
[15] G. Boyd, T. Rasing, R. Leite, Y. Shen (1984) Phys. Rev. B 30, 519-526.
[16] C. Chen, T. F. Heinz, D. Richard, Y. Shen (1981) Phys. Rev. Lett. 46, 1010-1012. Milan Mrksich and George M. Whitesides, “Using Self-Assembled Monolayers to Understand the Interactions of Man-Made Surfaces with Proteins and Cells,” Annu. Rev. Biophys. Biomol. Struct., 1996, 25:55-78.
V. G. Cheung et al., “Making and reading microarrays”, Nature Genetics (Suppl.), 1999, 21: 15-19.
L. A. Chrisey, G. U. Lee, C. E. O'Ferrall, “Covalent attachment of synthetic DNA to self-assembled monolayer films”.
Gabriel P. Lopez, Mark W. Albers, Stuart L. Schreiber, Reed Carroll, Ernest Peralta and George M. Whitesides, Convenient Methods for Patterning the Adhesion of Mammalian Cells to Surfaces Using Self-Assembled Monolayers of Alkanethiolates on Gold, J. Am. Chem. Soc., 1993, 115, 5877-5878.
Rahul Singhvi, Amit Kumar, gabriel P. Lopez, Gregory N. Stephanopoulos, Daniel I. C. Wang, George M. Whitesides and Donald E. Ingber, “Engineering Cell Shape and Function,” Science, vol. 264, Apr. 29, 1994, 696-698.
Levicky R, Herne T M, Tarlov M J, and Satija S K (1998). “Using Self-Assembly to Control the Structure of DNA Monolayers on Gold: A Neutron Reflectivity Study.” Journal of the American Chemical Society 120: 9787-9792.

1. A method for measuring an interaction at an interface between a probe and a target, said method comprising measuring an effect of said interaction between said probe and said target at said interface using a surface-selective nonlinear optical technique in the presence of indicators.

2. The method of claim 1 wherein said probes or targets are part of a surface or are attached to a surface.

3. The method of claim 1, wherein the nonlinear optical technique is second harmonic, sum frequency or difference frequency generation.

4. The method of claim 1, wherein said interaction is measured in the presence of a modulator, said modulator affecting the kinetic or equilibrium properties of said reactions, said modulator selected from the group comprising small molecules, drugs, agonists, inhibitors, blocking agents, or other components.

5. The method of claim 1, wherein said interfaces are comprised of cells, liposomes, beads or particles.

6. The method of claim 1, wherein said interactions comprise one or more of the following group: a binding reaction, a conformational change.

7. The method of claim 1, wherein said probes and targets comprise one or more components selected from the group consisting of nucleic acid, ligand, protein, small molecule, organic molecule, biological cell, virus, liposome, receptor, agonist, antibody, antigen, peptide, receptor, drug, blocking agent, enzyme, ligand, carbohydrate, nucleoside, oligosaccharide, organic molecule, toxin, oligonucleotide, polynucleotide, hormone, nucleic acid analog and peptide nucleic acid (PNA), ion channel receptor, G protein-coupled receptor (GPCR).

8. Nonlinear optical active indicators, said indicators comprising a moiety which possesses a hyperpolarizability.

9. The indicator according to claim 8, wherein the indicator includes a moiety selected from the group which comprises: Oxazole or oxadizole molecules 5-aryl-2-(4-pyridyl)oxazole 2-aryl-5-(4-pyridyl)oxazole 2-(4-pyridyl)cycloalkano[d]oxazoles Merocyanines Stilbenesa Indodicarbocyanines Hemicyanines Stilbazims Azo dyes Cyanines Stryryl-based dyes Methylene blue Diaminobenzene compounds Polyenes Diazostilbenes Tricyanovinyl aniline Tricyanovinyl azo Melamines Phenothiazine-stilbazole Polyimides Sulphonyl-substituted azobenzenes Indandione-1,3-pyidinium betaine Fluoresceins Benzooxazoles Perylenes Polymethacrylates Oxonols

10. The indicator of claim 8, wherein the indicator is a molecule or particle possessing a hyperpolarizability.
